(19) United States
(12) Patent Application Publication
(10) Pub. No.: US 2003/0165485 A1
(43) Pub. Date: Sep. 4, 2003

(54) Functional role and potential therapeutic use of Reelin, Gas6 and Protein S in relation to adult neural stem or progenitor cells

(76) Inventors: Goran Bertilsson, Vasterhaninge (SE); Anna Falk, Solna (SE); Jonas Frisen, Stockholm (SE); Jessica Heidrich, Arsta (SE); Kristina Hellstrom, Sodertalje (SE); Jarkko Kortesmaa, Stockholm (SE); Per Lindquist, Bromma (SE); Hanna Lundh, Solna (SE); Jacqueline McGuire, Huddinge (SE); Alex Mercer, Bromma (SE); Cesare Patrone, Hagersten (SE); Harriet Ronnholm, Trangsund (SE); Lilian Wikstrom, Spanga (SE); Olof Zachrisson, Spanga (SE)

Correspondence Address: Ivor R. Elrifi, Mintz, Levin, Cohn , Ferris,, Glovsky and Popeo, P.C., 666 Third Avenue, 24th Floor, New York, NY, 10017, US

(21) Appl. No.: 10/291,171

(22) Filed: Nov. 8, 2002

Related U.S. Application Data
(60) Provisional application No. 60/344,725, filed on Nov. 9, 2001.
(60) Provisional application No. 60/393,263, filed on Jul. 2, 2002.
(60) Provisional application No. 60/345,064, filed on Nov. 9, 2001.
(60) Provisional application No. 60/394,397, filed on Jul. 8, 2002.

(51) Int. Cl. A61K 38/46; A61K 39/395

(52) U.S. Cl. 424/94.6; 424/146.1

(57) ABSTRACT

The invention relates generally to methods of influencing central nervous system cells to produce progeny useful in the treatment of CNS disorders. More specifically, the invention includes methods of exposing a patient suffering from such a disorder to a reagent that modulates the proliferation, migration, differentiation and survival of central nervous system cells via Reelin, Gas6 or Protein S signaling. These methods are useful for reducing at least one symptom of the disorder.

### RELATED APPLICATIONS

This application claims the benefit of U.S. Ser. No. 60/344,725 filed Nov. 9, 2001; U.S. Ser. No. 60/393,263 filed Jul. 2, 2002; U.S. Ser. No. 60/345,064 filed Nov. 9, 2001; and 60/394,397 filed Jul. 8, 2002. The contents of these applications are incorporated herein by reference in their entirety.

### FIELD OF THE INVENTION

The invention relates generally to methods of influencing adult neural stem cells and neural progenitor cells to produce progeny that can replace damaged or missing neurons or other central nervous system (CNS) cell types. More specifically, the invention includes methods of exposing a patient suffering from a disorder to a reagent that regulates the differentiation, proliferation, survival and migration of central nervous system cells via modulation of Reelin, Gas6 or Protein S signaling. These methods are useful for reducing at least one symptom of a disorder.

### BACKGROUND OF THE INVENTION

For several years, it has been known that neural stem cells exist in the adult mammalian brain. This concept is of particular importance since the adult brain was thought to have very limited regenerative capacity. Moreover, the possibility to use adult-derived stem cells for tissue repair may help to overcome the ethical problems associated with embryonic cell research. Although the generation of neurons and glia can be observed in the adult brain, there is thus far only limited knowledge about stimulation of human neural stem cells in vitro and in vivo.

The first suggestions that new neurons were born in the adult mammalian brain came from studies performed in the 1960s (Altman and Das 1965; Altman and Das 1967). It however took another three decades and refined technical procedures to overthrow the dogma that neurogenesis within the mammalian CNS is restricted to embryogenesis and the perinatal period (for review see (Momma, Johansson et al. 2000); (Kuhn and Svendsen 1999)). Treatment of neural disease and injury traditionally focuses on keeping existing neurons alive, but possibilities now arise for exploiting neurogenesis for therapeutic treatments of neurological disorders and diseases.

The source of new neurons is neural stem cells (NSCs), located within the ependymal and/or subventricular zone (SVZ) lining the lateral ventricle (Doetsch, Callie et al. 1999; Johansson, Momma et al. 1999) and in the dentate gyrus of the hippocampus formation (Gage, Kempermann et al. 1998). Recent studies reveal the potential for several additional locations of NSC within the adult CNS (Palmer, Markakis et al. 1999). Asymmetric division of NSC maintain their number while generating a population of rapidly dividing precursor or progenitor cells (Johansson, Momma et al. 1999). The progenitors respond to a range of cues that dictate the extent of their proliferation and their fate, both in terms of the cell type that they differentiate into and the position that they ultimately take up in the brain.

The NSCs of the ventricular system in the adult are likely counterparts of the embryonic ventricular zone stem cells lining the neural tube whose progeny migrate away to form the CNS as differentiated neurons and glia (Jacobson 1991). NSCs persist in the adult lateral ventricle wall (LVW), generating neuronal progenitors which migrate down the rostral migratory stream to the olfactory bulb, where they differentiate into granule cells and periglomerular neurons (Lois and Alvarez-Buylla 1993). Substantial neuronal death occurs in the olfactory bulb generating a need for continuous replacement of lost neurons, a need satisfied by the migrating progenitors derived from the LVW (Biebl, Cooper et al. 2000). Further to this ongoing repopulation of olfactory bulb neurons, there are forceful indications that lost neurons from other brain regions can be replaced by progenitors from the LVW that differentiate into the lost neuron phenotype complete with appropriate neuronal projections and synapses with the correct target cell type (Snyder, Yoon et al. 1997; Magavi, Leavitt et al. 2000).

In vitro cultivation techniques have been established to identify the external signals involved in the regulation of NSC proliferation and differentiation (Johansson, Momma et al. 1999; Johansson, Svensson et al. 1999). The mitogens EGF and basic FGF allow neural progenitors, isolated from the ventricle wall and hippocampus, to be greatly expanded in culture (McKay 1997; Johansson, Svensson et al. 1999). The dividing progenitors remain in an undifferentiated state growing into large balls of cells known as neurospheres. Withdrawal of the mitogens combined with addition of serum induces differentiation of the progenitors into the three cell lineages of the brain: neurons, astrocytes and oligodendrocytes (Doetsch, Caille et al. 1999; Johansson, Momma et al. 1999). Application of specific growth factors can distort the proportions of each cell type in one way or another. For example, CNTF acts to direct the neural progenitors to an astrocytic fate (Johe, Hazel et al. 1996; Rajan and McKay 1998), while the thyroid hormone, triiodothyronine (T3) has been shown to promote oligodendrocyte differentiation (Johe, Hazel et al. 1996). Enhancement of neuronal differentiation of neural progenitors by PDGF has also been documented (Johe, Hazel et al. 1996; Williams, Park et al. 1997).

The ability to expand neural progenitor cells and then manipulate their cell fate has also had enormous implications in transplant therapies for neurological diseases in which specific cell types are lost. The most obvious example is Parkinson's Disease (PD) which is characterized by degeneration of dopaminergic neurons in the substantia nigra. Previous transplantation treatments for PD patients have used fetal tissue taken from the ventral midbrain at a time when substantia nigral dopaminergic neurons are undergoing terminal differentiation (Herman and Abrous 1994). The cells are grafted onto the striatum where they form synaptic contacts with host striatal neurons, their normal synaptic target, restoring dopamine turnover and release to normal levels with significant functional benefits to the patient (Herman and Abrous 1994) (for review see (Bjorklund and Lindvall 2000)). Grafting of fetal tissue is hindered by lack of donor tissue. In vitro expansion and manipulation of NSCs, however, can potentially provide a range of well characterized cells for transplant-based strategies for neurodegenerative diseases, such as dopaminergic cells for PD. To this aim, the identification of factors and pathways that govern the proliferation and differentiation of neural cell types will prove fundamental.

Ultimately the identification of these proliferative and differentiating factors is likely to provide insights into the stimulation of endogenous neurogenesis for the treatment of neurological diseases and disorders. Intraventricular infusion of both EGF and basic FGF have been shown to proliferate the ventricle wall cell population, and in the case of EGF, extensive migration of progenitors into the neighbouring striatal parenchyrna (Craig, Tropepe et al. 1996; Kuhn, Winkler et al. 1997). The progenitors differentiated predominantly into a glial lineage while reducing the generation of neurons (Kuhn, Winkler et al. 1997). A recent study found that intraventricular infusion of BDNF in adult rats stimulates an increase in the number of newly generated neurons in the olfactory bulb and rostral migratory stream, and in parenchymal structures, including the striatum, septum, thalamus and hypothalamus (Pencea, Bingaman et al. 2001). These studies demonstrate that the proliferation of progenitors within the SVZ of the LVW can be stimulated and that their lineage can be manipulated to neuronal and glial fates. Currently the number of factors known to affect neurogenesis in vivo is small and their effects are either undesired or limited.

Therefore, it is necessary to identify other factors that can selectively stimulate neural stem cell activity through proliferation of neural progenitors and differentiation into the desired neuronal cell type. This activity would be beneficial for both stimulation of in vivo neurogenesis and culture of cells for transplantation therapy. The present invention demonstrates a role for Reelin, Gas6, Protein S and their signaling pathways in the proliferation, differentiation, survival and migration of neural stem cells in vitro and in vivo.

### SUMMARY OF THE INVENTION

This invention relates generally to methods of influencing central nervous system cells to produce progeny that can replace damaged or missing neurons or other central nervous system cell types.

In one aspect, the invention includes a method of alleviating a symptom of a disease or disorder of the nervous system comprising administering Reelin, Gas6, Protein S, a molecule that regulates the phosphorylation status of Dab1 or a combination thereof to modulate neural stem cell or neural progenitor cell activity in vivo to a patient suffering from the disease or disorder of the nervous system. The symptom could be a phenotype or indicator of any of the diseases included in this invention. In a preferred embodiment of the invention, the neural stem cell or neural progenitor cell activity is proliferation, differentiation, migration or survival. In one embodiment of the invention, the activity regulated by Gas6 is proliferation, differentiation or survival. In another embodiment of the invention, the activity regulated by Reelin is proliferation. In a further embodiment of the invention, the activity regulated by Protein S is proliferation, differentiation or survival.

In all methods that involve the administration of Reelin, Gas6, Protein S, a molecule that regulates the phosphorylation status of Dab1 or combination thereof could be administered in an amount of 0.001 ng/kg/day to 10 mg/kg/day, preferably in an amount of 0.01 ng/kg/day to 5 mg/kg/day, more preferably in an amount of 0.1 ng/kg/day to 1 mg/kg/day and most preferably in an amount of 0.1 ng/kg/day to 1 μg/kg/day. In one embodiment of the invention, the Reelin, Gas6, Protein S, a molecule that regulates the phosphorylation status of Dab1 or combination thereof is administered to achieve a target tissue concentration of 0.01 nM to 30 nM. The tissue is selected from the group consisting of the volume adjacent to the lateral wall, hippocampus, alveus, striatum, substantia nigra, retina, nucleus basalis of Meynert, spinal cord and cortex or any region of tissue that is impaired by stroke injury or ischemic injury. In one embodiment of the invention, the Reelin, Gas6, Protein S, a molecule that regulates the phosphorylation status of Dab1 or a combination thereof is administered by injection, wherein the injection is given subcutaneously, intraperitoneally, intramusclularly, intracerebroventricularly, intraparenchymally, intrathecally or intracranially. In another embodiment of the invention, the Reelin, Gas6, Protein S, a molecule that regulates the phosphorylation status of Dab1 or combination thereof is administered orally. In an further embodiment of the invention, the Reelin, Gas6, Protein S, a molecule that regulates the phosphorylation status of Dab1 or a combination thereof is administered to the buccal, nasal, rectal mucosa or they are administered via peptide fusion or micelle delivery.

In all embodiment of the inventions, the reference to disease or disorder of the nervous system is selected from the group consisting of neurodegenerative disorders, neural stem cell disorders, neural progenitor disorders, ischemic disorders, neurological traumas, affective disorders, neuropsychiatric disorders, degenerative diseases of the retina, retinal injury/trauma and learning and memory disorders. In one embodiment of the invention, the disease or disorder of the nervous system is selected from the group consisting of Parkinson's disease and Parkinsonian disorders, Huntington's disease, Alzheimer's disease, Amyotrophic Lateral Sclerosis, spinal ischemia, ischemic stroke, spinal cord injury and cancer-related brain/spinal cord injury. In a further embodiment of the invention, the disease or disorder of the nervous system is selected from the group consisting of schizophrenia and other psychoses, lissencephaly syndrome, depression, bipolar depression/disorder, anxiety syndromes/disorders, phobias, stress and related syndromes, cognitive function disorders, aggression, drug and alcohol abuse, obsessive compulsive behaviour syndromes, seasonal mood disorder, borderline personality disorder, cerebral palsy, life style drug, multi-infarct dementia, Lewy body dementia, age related/geriatric dementia, epilepsy and injury related to epilepsy, temporal lobe epilepsy, spinal cord injury, brain injury, trauma related brain/spinal cord injury, anti-cancer treatment related brain/spinal cord tissue injury, infection and inflammation related brain/spinal cord injury, environmental toxin related brain/spinal cord injury, multiple sclerosis, autism, attention deficit disorders, narcolepsy and sleep disorders.

In another aspect, the invention provides a method of modulating a Reelin receptor, Gas 6 receptor, a Protein S receptor or a combination thereof, on a neural stem cell or neural progenitor cell, the method comprising exposing the cell expressing the receptor to exogenous reagent, antibody, or affibody, wherein the exposure induces or inhibits the neural stem cell or neural progenitor cell to proliferate, differentiate or survive. In one embodiment of the invention, the Reelin, Gas6 or Protein S receptor is a fragment of the full length protein. This invention also contemplates proteins with point mutations and post-translational modifications, such as gamma-carboxylation of Gas6 and Protein S, that provide the same function as wild-type receptors. In another embodiment of the invention, the reagent is Reelin, Gas6, Protein S or a molecule that regulates the phosphorylation status of Dab1. The antibody could be a monoclonal or a polyclonal antibody. In another embodiment of the invention, the neural stem cell or neural progenitor cell is derived from fetal brain, adult brain, neural cell culture, a neurosphere, tissue enclosed by dura mater, peripheral nerves, ganglia, pancreas, skin, muscle, adult bone marrow, umbilical cord tissue and umbilical cord blood.

In a further aspect, the invention includes a method for reducing a symptom of a disease or disorder of the central nervous system in a mammal in need of such treatment comprising administering Reelin, Gas6, Protein S, a molecule that regulates the phosphorylation status of Dab1 or a Reelin, Gas6 or Protein S agonist or antagonist to the mammal. In one embodiment of the invention, the Reelin, Gas6, Protein S, a molecule that regulates the phosphorylation status of Dab1 or a Reelin, Gas6 or Protein S agonist or antagonist is adminstered in an amount of 0.001 ng/kg/day to 10 mg/kg/day, preferably in an amount of 0.01 ng/kg/day to 5 mg/kg/day, more preferably in an amount of 0.1 ng/kg/day to 1 mg/kg/day and most preferably in an amount of 0.1 ng/kg/day to 1 μg/kg/day. In another embodiment of the invention, the Reelin, Gas6 or Protein S agonist or antagonist is selected from the group consisting of an antibody, an affibody, a small molecule and a receptor. The administration could be local or systemic.

In another embodiment of the invention, the methods described could further comprise administering a ventricle wall permeability enhancer. In a preferred embodiment of the invention, the ventricle wall permeability enhancer is administered before, during or after administration of Reelin, Gas6, Protein S or a molecule that regulates the phosphorylation status of Dab1 or a Reelin, Gas6 or Protein S agonist or antagonist. In a further embodiment of the invention, the ventricle wall permeability enhancer or the Reelin, Gas6, Protein S, molecule that regulates the phosphorylation status of Dab1 or Reelin, Gas6 or Protein S agonist or antagonist are admixed with a pharmaceutically acceptable carrier. The methods described in the invention could also further comprise administration of one or more agents selected from the group consisting of stem cell mitogens, survival factors, glial-lineage preventing agents, anti-apoptotic agents, anti-stress medications, neuroprotectants, anti-pyrogenics, differentiation factors and a combination thereof.

In another aspect, the invention provides a method for inducing the in situ proliferation, differentiation or survival of a neural stem cell or neural progenitor cell located in the neural tissue of a mammal, the method comprising administering a therapeutically effective amount of Reelin, Gas6, Protein S or a molecule that regulates the phosphorylation status of Dab1 to the neural tissue to modulate the proliferation, differentiation or survival of the cell. In one embodiment of the invention, a reagent is used to modulate the proliferation, differentiation or survival of the cell. The reagent is selected from the group consisting of an antibody, an affibody, a small molecule and a receptor. In one embodiment of the invention, the administration of the Reelin, Gas6, Protein S or molecule that regulates the phosphorylation status of Dab1 is systemic or local. In another embodiment of the invention, the administration of the Reelin, Gas6, Protein S or a molecule that regulates the phosphorylation status of Dab1 alleviates a symptom of a diseases or disorders of the nervous system.

In another aspect, the invention includes a method for accelerating the growth of neural stem cells or neural progenitor cells in a desired target tissue in a subject, comprising administering to the subject an expression vector containing a Reelin, Gas6 or Protein S gene in a therapeutically effective amount. In a preferred embodiment of the invention, the expression vector is administered by injection, wherein the injection is given subcutaneously, intraperitoneally, intramuscluarly, intracerebroventricularly, intraparenchymally, intrathecally or intracranially. In another embodiment of the invention, the expression vector is administered orally, via peptide fusion or micelle delivery to the buccal, nasal or rectal mucosa. In another embodiment of the invention, the expression vector is a non-viral expression vector encapsulated in a liposome.

In another aspect, the invention includes a method of enhancing neurogenesis in a patient suffering from a disease or disorder of the central nervous system, by infusion of Reelin, Gas6, Protein S, a molecule that regulates the phosphorylation status of Dab1 or a Reelin, Gas6 or Protein S receptor agonist or antagonist. In a preferred embodiment of the invention, the infusion is selected from the group consisting of intraventricular, intravenous, sublingual, subcutaneous and intraarterial infusion.

In a further aspect, the invention provides a method of alleviating a symptom in a patient suffering from a disease or disorder of the central nervous system by enhancing neurogenesis through infusion of Reelin, Gas6, Protein S, a molecule that regulates the phosphorylation status of Dab1 or a Reelin, Gas6 or Protein S receptor agonist or antagonist.

In another aspect,the invention provides a method for producing a population of cells enriched for human neural stem cells or human neural progenitor cells, comprising: (a) contacting a population containing neural stem cells or neural progenitor cells with a reagent that recognizes a determinant on a Reelin receptor, Gas6 receptor or Protein S receptor; and (b) selecting for cells in which there is contact between the reagent and the determinant on the surface of the cells of step (a), to produce a population highly enriched for central nervous system stem cells. In one embodiment of the invention, the reagent selected from the group consisting of a soluble receptor, a small molecule, a peptide, an antibody and an affibody. In another embodiment of the invention, the soluble receptor is a Reelin, Gas6 or Protein S receptor.

In a further aspect, the invention includes an in vitro cell culture comprising a cell population generated by the method previously described wherein the cell population is enriched in receptor expressing cells wherein the receptors are selected from the group consisting of VLDLR, ApoER2, Axl, Tyro3 and Mer receptor.

In one aspect, the invention includes a method for alleviating a symptom of a disease or disorder of the central nervous system comprising administering the population of cells described above to a mammal in need thereof. In a further aspect, the invention includes a non-human mammal engrafted with the human neural stem cells or neural progenitor cells previously described. In a preferred embodiment of the invention, the non-human mammal is selected from the group including rat, mouse, rabbit, horse, sheep, pig and guinea pig.

In another aspect, the invention includes a method of reducing a symptom of a disease or disorder of the central nervous system in a subject comprising the steps of administering into the spinal cord of the subject a composition comprising a population of isolated neural stem cells or neural progenitor cells obtained from fetal or adult tissue; and Reelin, Gas6, Protein S, a molecule that regulates the phosphorylation status of Dab1 or a Reelin, Gas6 or Protein S agonist or antagonist or a combination thereof such that the symptom is reduced.

In another aspect, the invention includes a method of gene delivery and expression in a target cell of a mammal, comprising the step of introducing a viral vector into the target cell, wherein the viral vector has at least one insertion site containing a nucleic acid which encodes Reelin, Gas6, Protein S, a molecule that regulates the phosphorylation status of Dab1, a Reelin receptor, a Gas6 receptor or a Protein S receptor, the nucleic acid gene operably linked to a promoter capable of expression in the host. In one embodiment of the invention, the viral vector is a non-lytic viral vector.

In another aspect, the invention includes a method of gene delivery and expression in a target cell of a mammal comprising the steps of: (a) providing an isolated nucleic acid fragment of a nucleic acid sequence which encodes for Reelin, Gas6, Protein S, Dab1, a molecule that regulates the phosphorylation status of Dab1, a Reelin receptor, a Gas6 receptor or a Protein S receptor; (b) selecting a viral vector with at least one insertion site for insertion of the isolated nucleic acid fragment operably linked to a promoter capable of expression in the target cells; (c) inserting the isolated nucleic acid fragment into the insertion site, and (d) introducing the vector into the target cell wherein the gene is expressed at detectable levels. In one embodiment of the invention, the virus is selected from the group consisting of retrovirus, adenovirus, pox virus, iridoviruses, coronaviruses, togaviruses, caliciviruses, lentiviruses, adeno-associated viruses and picornaviruses. In another embodiment of the invention, the pox virus is vaccinia. In another embodiment of the invention, the virus is a strain that has been genetically modified or selected to be non-virulent in a host.

In a further aspect, the invention includes a method for alleviating a symptom of a disease or disorder of the central nervous system in a patient comprising the steps of: (a) providing a population of neural stem cells or neural progenitor cells; (b) suspending the neural stem cells or neural progentor cells in a solution comprising a mixture comprising Reelin, Gas6, Protein S or a molecule that regulates the phosphorylation status of Dab1 to generate a cell suspension; (c) delivering the cell suspension to an injection site in the central nervous system of the patient to alleviate the symptom. In one embodiment of the invention, the method described further comprises the step of injecting the injection site with a growth factor for a period of time before the step of delivering the cell suspension. In another embodiment of the invention, the method described further comprises the step of injecting the injection site with the growth factor after said delivering step.

In a further aspect, the invention includes a method for transplanting a population of cells enriched for human neural stem cells or human neural progenitor cells, comprising: (a) contacting a population containing neural stem cells or neural progenitor cells with a reagent that recognizes a determinant on a Reelin receptor, Gas6 receptor or Protein S receptor; (b) selecting for cells in which there is contacted between the reagent and the determinant on the surface of the cells of step (a), to produce a population highly enriched for central nervous system stem cells; and (c) implanting the selected cells of step (b) into a non-human mammal.

In a further aspect, the invention includes a method of modulating Reelin, Gas6 or Protein S receptor or a Reelin, Gas6 or Protein S ligand on the surface of a neural stem cell or neural progenitor cell comprising the step of exposing the cell expressing the receptor, or ligand to exogenous reagent, antibody, or affibody, wherein the exposure induces or inhibits the neural stem cell or neural progenitor cell to proliferation, differentiation or survival. In one embodiment of the invention the neural stem cell or neural progenitor cell is derived from fetal brain, adult brain, neural cell culture or a neurosphere.

In a further aspect, the invention includes a method of determining an isolated candidate Reelin, Gas6 or Protein S receptor modulator compound for its ability to modulate neural stem cell or neural progenitor cell activity comprising the steps of: (a) administering the isolated candidate compound to a non-human mammal; and (b) determining if the candidate compound has an effect on modulating the neural stem cell or neural progenitor cell activity in the non-human mammal. In a preferred embodiment of the invention, the determining step comprises comparing the neurological effects of said non-human mammal with a referenced non-human mammal not administered the candidate compound. In a further embodiment of the invention, the compound is selected from the group consisting of a peptide, a small molecule, a soluble receptor, a receptor agonist and a receptor antagonist. In another embodiment of the invention, the compound is selected from the group consisting of VLDLR, ApoER2, Axl, Mer, Tyro3, a soluble fragment thereof and an extracellular fragment thereof. The neural stem cell or neural progenitor cell activity could be proliferation, differentiation, migration or survival. In another embodiment of the invention, the Reelin, Gas6 or Protein S receptor modulator is administered by injection, wherein the injection is given subcutaneously, intraperitoneally, intramuscluarly, intracerebroventricularly, intraparenchymally, intrathecally or intracranially. In another embodiment of the invention, the Reelin, Gas6 or Protein S receptor modulator is administered via peptide fusion or micelle delivery.

### BRIEF DESCRIPTION OF THE DRAWINGS

FIG. 1 illustrates Reelin, VLDLR, ApoER2 and Dab1 mRNA expression in cultured non-adherent mouse neurospheres (A) and adult human neural stem cell cultures (B) by RT-PCR.

FIG. 2 illustrates brightfield micrographs of VLDLR mRNA in coronal sections of adult mouse brain.

FIG. 3 illustrates darkfield microphotographs of reelin mRNA in coronal sections of adult mouse brain.

FIG. 4 illustrates darkfield microphotographs of dab1 mRNA in coronal sections of adult mouse brain.

FIG. 5 illustrates immunohistochemistry of wild-type and Reeler brains after two weeks of BrdU treatment.

FIG. 6 illustrates expression of Gas6, Tyro3, Axl and Mer in cultured non-adherent mouse neurospheres.

FIG. 7 illustrates mRNA expression of Gas6, Axl and Tyro3 in cultured adult human neural stem cells.

FIG. 8 illustrates mRNA expression of Gas6, Tyro3 and Axl in neurogenic regions of the adult mouse brain.

FIG. 9 illustrates Tyro3, Axl and Mer protein levels in cultured non-adherent mouse neurospheres.

FIG. 10 illustrates the effect of Gas6 on proliferation of cultured non-adherent mouse neurospheres.

FIG. 11 illustrates the effect of Gas6 on proliferation of adherent mouse neural stem cells/progenitor cultures.

FIG. 12 illustrates the effect of Gas6 on induction of tyrosine phosphorylation of adherent mouse neural stem cells/progenitor cultures.

FIG. 13 illustrates that exposure to Gas6 results in a proportional increase in β-III tubulin positive cells in Gas6 treated versus control treated cells.

FIG. 14 illustrates the effect of Gas6 on the number of BrdU positive cells in the subventricular zone.

FIG. 15 illustrates the effect of Gas6 on the number of BrdU positive cells in the subventricular zone.

### DETAILED DESCRIPTION OF THE INVENTION

It has been discovered that certain reagents are capable of modulating the differentiation, migration, proliferation and survival of neural stem/progenitor cells both in vitro and in vivo. As used herein, the term “modulate” refers to having an affect in such a way as to alter the differentiation, migration, proliferation and survival of neural stem cell (NSC) or neural progenitor cell (NPC) activity. Since undifferentiated, pluripotent stem cells can proliferate in culture for a year or more, the invention described in this disclosure provides an almost limitless supply of neural precursors.

As used herein, the term “neural stem cells” (NSCs) can be identified by their ability to undergo continuous cellular proliferation, to regenerate exact copies of themselves (self-renew), to generate a large number of regional cellular progeny, and to elaborate new cells in response to injury or disease. The terms “neural progenitor cells” or “neural precursor cells” (NPCs) mean cells that can generate progeny that are either neuronal cells (such as neuronal precursors or mature neurons) or glial cells (such as glial precursors, mature astrocytes, or mature oligodendrocytes). Typically, the cells express some of the phenotypic markers that are characteristic of the neural lineage. They also do not usually produce progeny of other embryonic germ layers when cultured by themselves in vitro unless dedifferentiated or reprogrammed in some fashion. As used herein, the term “neurosphere” refers to the ball of cells consisting of NSCs and NPCs.

As used herein, the term “reagent” refers to any substance that is chemically and biologically capable of activating a receptor, including peptides, small molecules, antibodies (or fragments thereof), affibodies and any molecule that dimerizes or multimerizes the receptors or replaces the need for activation of the extracellular domains. In one embodiment, the reagent is a small molecule.

As used herein, the term “antibody” as used in this disclosure refers to both polyclonal and monoclonal antibody. The ambit of the term deliberately encompasses not only intact immunoglobulin molecules, but also such fragments and derivatives of immunoglobulin molecules (such as single chain Fv constructs, diabodies and fusion constructs) as may be prepared by techniques known in the art, and retaining a desired antibody binding specificity. The term “affibody” (U.S. Pat. No. 5,831,012) refers to highly specific affinity proteins that can be designed to bind to any desired target molecule. These antibody mimics can be manufactured to have the desired properties (specificity and affinity), while also being highly robust to withstand a broad range of analytical conditions, including pH and elevated temperature. The specific binding properties that can be engineered into each capture protein allow it to have very high specificity and the desired affinity for a corresponding target protein. A specific target protein will thus bind only to its corresponding capture protein. The small size (only 58 amino acids), high solubility, ease of further engineering into multifunctional constructs, excellent folding and absence of cysteines, as well as a stable scaffold that can be produced in large quantities using low cost bacterial expression systems, make affibodies superior capture molecules to antibodies or antibody fragments, such as Fab or single chain Fv (scFv) fragments, in a variety of Life Science applications.

Preferred reagents of the invention include Reelin, Gas6, Protein S and any molecule that regulates the phosphorylation status of disabled1 (Dab1). The invention provides a method for in vivo modulation of Reelin and Gas6 activity and for therapeutic administration of Reelin and Gas6 and drug screening. In one embodiment, Reelin, Gas6, ProteinS or any molecule that regulates the phosphorylation status of Dab1 or their agonist are administered to neural tissue. In a preferred embodiment, the neural tissue is fetal or adult brain. In yet another embodiment, the population containing neural or neural-derived cells is obtained from a neural cell culture or neurosphere.

Receptors for the invention include members of the Reelin receptors, members of the Gas6 receptors and members of the Protein S receptors. Such receptors may include: VLDLR, ApoER2, Tyro3, Axl and Mer receptor.

Reelin Receptors and Their Ligands

Reelin is a secreted protein produced by distinct cell types in the developing brain, including Cajal-Retzius (CR) cells in the cerebral cortex and hippocampus and granule cells in the cerebellum (D'Arcangelo, Miao et al. 1995; Ogawa, Miyata et al. 1995). The reelin gene was found to be disrupted in the classical neurological mutant mouse, reeler, characterized by inappropriate neuronal positioning in developing brain structures (D'Arcangelo, Miao et al. 1995). Behavioural defects, such as ataxia, temors, imbalance and a reeling gait are associated with neuronal ectopia in laminated brain regions, including the cerebral and cerebellar cortices and hippocampus. There is also severe hypoplasia of cerebellum (D'Arcangelo and Curran 1998). The role of Reelin has been best characterized with regard to its role in cell positioning in the developing brain. In the developing cerebral cortex, neurons migrate through the subplate layer and come to rest below CR neurons in the marginal zone. Each subsequent cortical neuron migrates past its predecessors, coming to rest immediately below the CR neurons. In the reeler mutants, however, the migrating neurons fail to penetrate the subplate layer and stack up behind their predecessors forming “an inside out” layering of the cerebral cortex (for review see (D'Arcangelo, Homayouni et al. 1999).

An indistinguishable phenotype from that of the reeler mouse was observed in other mutant mice, carrying mutations in the disabled-1 (dab1) gene (Howell, Hawkes et al. 1997; Sheldon, Rice et al. 1997), and where both the very low density lipoprotein receptor (VLDLR) and apolipoprotein E receptor 2 (ApoER2) are knocked out (Trommsdorff, Gotthardt et al. 1999). The VLDLR and ApoER2 are members of the LDLR family. While all members bind apolipoprotein E containing lipoproteins, the functions of VLDLR and ApoER2 are not so well defined. VLDLR and ApoER2 bind and internalize a variety of extracellular ligands including proteases, protease inhibitors, peptide hormones, and vitamin carrier proteins (for review see (Nimpf and Schneider 2000)), however, the pronounced phenotype of the double knockout suggested an all together new role for these receptors. Further biochemical data confirmed the suggestions from the reeler, mutant dab1 and vldlr/apoER2 knockout mice that a signaling pathway exists involving the extracellular protein reelin, binding directly to VLDLR and ApoER2, and signaling through phosphorylation of the non-receptor tyrosine kinase, Dab1, that underpins neuronal positioning during development (D'Arcangelo, Homayouni et al. 1999).

Following the completion of neuronal migration, the proteins of the Reelin signaling pathway (Reelin, VLDLR, ApoER2, Dab1) continue to be expressed into postnatal and adult life. Indeed, the expression pattern of Reelin mRNA is drastically altered in the adult as compared to developmental stages, with high expression in hippocampus, entorhinal cortex, olfactory bulb and cerebellum (Alcantara, Ruiz et al. 1998). Recent evidence lends credence to the notion that the Reelin pathway contributes to the formation of neuronal circuits in the CNS (Rice, Nusinowitz et al. 2001) and the guidance mechanisms governing the formation of hippocampal projections (Borrell, Del Rio et al. 1999). Involvement in neurodegeneration has also been proposed based on the interference of VLDLR and ApoER2 binding to Reelin by ApoE, which accumulates to very high levels in response to neuronal injury, and of which the type 4 is a susceptibility factor for Alzheimer's Disease (AD). In addition, Reelin expression is down-regulated in schizophrenic patients and patients suffering major depression suggesting a role in the pathogenesis of these diseases (Fatemi, Earle et al. 2000).

Altered expression profiles of Reelin and members of its pathway have been observed following hypoxia in the developing mouse brain that could correlate a function in ischemia (Curristin, Cao et al. 2001). While indications are becoming clear that the Reelin pathway plays some part in a number of diseases, only the ApoER2 has been implicated in brain diseases functioning outside this defined pathway. ApoER2 is linked to AD through its potential to bind α₂-macroglobulin (α₂M) (Stockinger, Hengstschlager-Ottnad et al. 1998), which in turn associates with the β-amyloid peptide (Hughes, Khorkova et al. 1998), both of which are present in neuritic plaques of AD patients (Rebeck, Harr et al. 1995). VLDLR on the other hand has an intriguing side with respect to proliferation of endothelial cells. Tissue factor pathway inhibitor (TFPI) has been identified as a ligand for VLDLR and on binding the receptor potently inhibits proliferation of basic FGF-stimulated endothelial cell growth (Hembrough, Ruiz et al. 2001). Indeed, this ligand-receptor system has been proposed as a target for the development of anti-angiogenic and anti-tumor agents (Hembrough, Ruiz et al. 2001). Based on recent studies regarding the role of the Reelin pathway in the adult (Rice, Nusinowitz et al. 2001) (Borrell, Del Rio et al. 1999) (Fatemi, Earle et al. 2000) (Curristin, Cao et al. 2001), it is clear that Reelin has additional functions in the adult as compared to its role during brain development.

The Axl Subfamily of Receptor Protein Tyrosine Kinases

Receptor protein tyrosine kinases (RPTKs) are a family of transmembrane spanning receptors endowed with intrinsic, ligand-stimulatable protein tyrosine kinase (PTK) activity (Blume-Jensen and Hunter 2001). Upon binding to their corresponding ligands, these receptors initiate a complex series of intracellular reactions which ultimately result in diverse cellular responses such as proliferation, differentiation, cell motility, quiescence or apoptosis. Moreover, RPTKs have been shown to play important roles in the developing nervous system, the best characterized of which are the Trks which, together with their ligands, the neurotrophins, have been shown to play a role in the regulation of neuronal cell number by promoting cell survival (Bothwell 1995; Henderson 1996). Furthermore, a distinct subfamily of the RPTKs, the Eph receptors and their ligands, the ephrins, aid in the establishment of topographic maps, best studied in the retinotectal projection (Flanagan and Vanderhaeghen 1998; O'Leary and Wilkinson, 1999). In contrast however, the role of RPTKs in the adult mammalian CNS is less well documented although Trks have been implicated in modulating neural plasticity (McAllister et al., 1999)

Tyro3, together with Axl and Mer comprise a distinct subclass of RPTKs collectively known as the Axl subfamily of RPTKs. Each member of this subfamily has been identified under different names by various investigators. Hence Tyro3 has been designated Dtk, etk2, brt, Rse, sky and tif; Axl has been identified as Ufo, Tyro7 and Ark and Mer is also known as Tyro12, eyk and Nyk (Reviewed in Prieto et al., 1999; 2000). Members of this subfamily are distinguished on the basis of significant amino acid similarities in their extracellular regions resulting in a common domain structure characterized by the presence of two immunoglobulin (Ig)-like domains located N-terminal to two fibronectin type III repeats. Interestingly, Ig-like and FN type III repeats are commonly found in many cell surface receptors, neural cell adhesion molecules (NCAMs) and extracellular matrix proteins, suggesting that members of this subfamily may represent a novel class of cell surface receptors that mediate cell adhesive interactions. In support of this model, Axl has been shown to function as a homophilic cell adhesion molecule (CAM) inducing cell aggregation in cultured fibroblasts resulting in an increase in receptor phosphorylation (Bellosta et al., 1995). In addition, upon binding specific ligand, Axl has also been shown to function as a heterophilic CAM (McCloskey et al., 1997; Nakano et al., 1997).

Members of the Axi subfamily of RPTKs can be activated by a common ligand, Gas6 (growth arrest specific gene-6), a member of a class of vitamin K dependent proteins, which upon binding, induces receptor phosphorylation. (Ohashi et al., 1995; Vamum et al., 1995; Mark et al., 1996; Nagata et al., 1996; Chen et al., 1997). Gas6 was originally isolated as a growth arrest specific gene whose expression is up-regulated during quiescence in fibroblasts (Manfioletti et al., 1993). With respect to ligand binding, members of this subfamily display a distinct hierarchy, with highest affinity displayed by Axl followed by Tyro3 and finally Mer. Thus Gas6 may be considered a central factor with the potential to modulate distinct networks of target genes such that colocalization of more than one family member may result in different signaling responses upon binding of the same ligand. Interestingly, Gas6 displays significant amino acid sequence identity to Protein S, an additional member of the vitamin K-dependent family and an important co-factor in the inhibition of the blood coagulation cascade, suggesting possible functional similarities between these two proteins (Manfioletti at al., 1993). In support of this model, Protein S has been proposed to serve as an additional ligand for Tyro3 (Stitt et al., 1995).

Tissue specific expression analysis has shown that both Axl and Mer are widely expressed in adult mammalian tissues. Similarly, Gas6 has also been shown to be expressed ubiquitously (Manfioletti et al., 1993). In contrast, Tyro3 displays a highly tissue restricted pattern of expression with predominant expression in the brain and low levels in the testis and ovary. The initial identification of Gas6 as a protein expressed in response to growth arrest in fibroblasts suggested that it may function as a negative regulator of cell proliferation (Manfioletti et al., 1993). The observation that overexpression of Axl and Tyro has transforming capacity suggests however, that it may function as a growth factor in certain cell types.

Role of Gas6 and Axl Subfamily of RPTKs in the Adult Mammalian CNS

Regulated expression of Gas6, Axl and Tyro3 has been observed in response to nerve injury (Li et al., 1996 and references therein). Interestingly, this has also been observed for Protein S. More recently, a role for Gas6 in attenuating hippocampal neural cell death in response to serum starvation in vitro has been demonstrated (Funakoshi et al., 2002). Furthermore, the potential importance of this class of receptors in protection from apoptosis in the central nervous system has been further underscored by the fact that mice in which all three receptors, Tyro3, Axl and Mer, have been knocked out present substantial cell death in the a number of regions in the adult brain including the hippocampus, cerebellum and neocortex (Lu et al., 1999). Previous studies have shown that Gas6, together with its cognate receptors Tyro3, Axl and Mer are widely expressed in the brain of postnatal rats (Prieto et al., 1999; 2000). In comparison to Axl and Mer, Tyro3 has been shown to be more highly expressed particularly in the dendritic compartment of cortical and hippocampal (CA1) pyramidal neurons. Moreover, Gas6 has also been shown to colocalize with Tyro-3 in specific subsets of neurons within these regions, suggesting potential autocrine roles. Thus, Tyro3 may serve as the primary receptor for Gas6 activated signal transduction in the adult mammalian CNS (Funakoshi, H. et al., 2002).

Production of Reagents

Reagents for treatment of patients are recombinantly produced, purified and formulated according to well known methods.

Reagents of the invention and individual moieties or analogs and derivatives thereof, can be chemically synthesized. A variety of protein synthesis methods are common in the art, including synthesis using a peptide synthesizer. See, e.g., Peptide Chemistry, A Practical Textbook, Bodasnsky, Ed. Springer-Verlag, 1988; Merrifield, Science 232: 241-247 (1986); Barany, et al, Intl. J. Peptide Protein Res. 30: 705-739 (1987); Kent, Ann. Rev. Biochem. 57:957-989 (1988), and Kaiser, et al, Science 243: 187-198 (1989). The peptides are purified so that they are substantially free of chemical precursors or other chemicals using standard peptide purification techniques. The language “substantially free of chemical precursors or other chemicals” includes preparations of peptide in which the peptide is separated from chemical precursors or other chemicals that are involved in the synthesis of the peptide. In one embodiment, the language “substantially free of chemical precursors or other chemicals” includes preparations of peptide having less than about 30% (by dry weight) of chemical precursors or non-peptide chemicals, more preferably less than about 20% chemical precursors or non-peptide chemicals, still more preferably less than about 10% chemical precursors or non-peptide chemicals, and most preferably less than about 5% chemical precursors or non-peptide chemicals.

Chemical synthesis of peptides facilitates the incorporation of modified or unnatural amino acids, including D-amino acids and other small organic molecules. Replacement of one or more L-amino acids in a peptide with the corresponding D-amino acid isoforms can be used to increase the resistance of peptides to enzymatic hydrolysis, and to enhance one or more properties of biologically active peptides, e.g., receptor binding, functional potency or duration of action. See, e.g., Doherty, et al., 1993. J. Med. Chem. 36: 2585-2594; Kirby, et al., 1993, J. Med. Chem. 36:3802-3808; Morita, et al., 1994, FEBS Lett. 353: 84-88; Wang, et al., 1993 Int. J. Pept. Protein Res. 42: 392-399; Fauchere and Thiunieau, 1992. Adv. Drug Res. 23: 127-159.

Introduction of covalent cross-links into a peptide sequence can conformationally and topographically constrain the peptide backbone. This strategy can be used to develop peptide analogs of reagents with increased potency, selectivity and stability. A number of other methods have been used successfully to introduce conformational constraints into peptide sequences in order to improve their potency, receptor selectivity and biological half-life. These include the use of (i) C_{α}-methylamino acids (see, e.g., Rose, et al., Adv. Protein Chem. 37: 1-109 (1985); Prasad and Balaram, CRC Crit. Rev. Biochem., 16: 307-348 (1984)); (ii) N_{α}-methylamino acids (see, e.g., Aubry, et al., Int. J. Pept. Protein Res., 18: 195-202 (1981); Manavalan and Momany, Biopolymers, 19: 1943-1973 (1980)); and (iii) α,β-unsaturated amino acids (see, e.g., Bach and Gierasch, Biopolymers, 25: 5175-S192 (1986); Singh, et al., Biopolymers, 26: 819-829 (1987)). These and many other amino acid analogs are commercially available, or can be easily prepared. Additionally, replacement of the C-terminal acid with an amide can be used to enhance the solubility and clearance of a peptide.

Alternatively, a reagent may be obtained by methods well-known in the art for recombinant peptide expression and purification. A DNA molecule encoding the protein reagent can be generated. The DNA sequence is known or can be deduced from the protein sequence based on known codon usage. See, e.g., Old and Primrose, Principles of Gene Manipulation 3^{rd} ed., Blackwell Scientific Publications, 1985; Wada et al., Nucleic Acids Res. 20: 2111-2118(1992). Preferably, the DNA molecule includes additional sequence, e.g., recognition sites for restriction enzymes which facilitate its cloning into a suitable cloning vector, such as a plasmid. Nucleic acids may be DNA, RNA, or a combination thereof. Nucleic acids encoding the reagent may be obtained by any method known within the art (e.g., by PCR amplification using synthetic primers hybridizable to the 3′- and 5′-termini of the sequence and/or by cloning from a cDNA or genomic library using an oligonucleotide sequence specific for the given gene sequence, or the like). Nucleic acids can also be generated by chemical synthesis.

Any of the methodologies known within the relevant art regarding the insertion of nucleic acid fragments into a vector may be used to construct expression vectors that contain a chimeric gene comprised of the appropriate transcriptional/translational control signals and reagent-coding sequences. Promoter/enhancer sequences within expression vectors may use plant, animal, insect, or fungus regulatory sequences, as provided in the invention.

A host cell can be any prokaryotic or eukaryotic cell. For example, the peptide can be expressed in bacterial cells such as E. coli, insect cells, fungi or mammalian cells (such as Chinese hamster ovary cells (CHO) or COS cells). Other suitable host cells are known to those skilled in the art. In one embodiment, a nucleic acid encoding a reagent is expressed in mammalian cells using a mammalian expression vector. Examples of mammalian expression vectors include pCDM8 (Seed (1987) Nature 329:840) and pMT2PC (Kaufman et al. (1987) EMBO J 6: 187-195).

The host cells, can be used to produce (e.g., overexpress) peptide in culture. Accordingly, the invention further provides methods for producing the peptide using the host cells of the invention. In one embodiment, the method comprises culturing the host cell of invention (into which a recombinant expression vector encoding the peptide has been introduced) in a suitable medium such that peptide is produced. The method further involves isolating peptide from the medium or the host cell. Ausubel et al., (Eds). In: Current Protocols in Molecular Biology. J. Wiley and Sons, New York, NY. 1998.

An “isolated” or “purified” recombinant peptide or biologically active portion thereof is substantially free of cellular material or other contaminating proteins from the cell or tissue source from which the peptide of interest is derived. The language “substantially free of cellular material” includes preparations in which the peptide is separated from cellular components of the cells from which it is isolated or recombinantly produced. In one embodiment, the language “substantially free of cellular material” includes preparations of peptide having less than about 30% (by dry weight) of peptide other than the desired peptide (also referred to herein as a “contaminating protein”), more preferably less than about 20% of contaminating protein, still more preferably less than about 10% of contaminating protein, and most preferably less than about 5% contaminating protein. When the peptide or biologically active portion thereof is recombinantly produced, it is also preferably substantially free of culture medium, e.g., culture medium represents less than about 20%, more preferably less than about 10%, and most preferably less than about 5% of the volume of the peptide preparation.

The invention also pertains to variants of a reagent that function as either agonists (mimetics) or as antagonists. Variants of a reagent can be generated by mutagenesis, e.g., discrete point mutations. An agonist of a reagent can retain substantially the same, or a subset of, the biological activities of the naturally occurring form of the reagent. An antagonist of the reagent can inhibit one or more of the activities of the naturally occurring form of the reagent by, for example, competitively binding to the receptor. Thus, specific biological effects can be elicited by treatment with a variant with a limited function. In one embodiment, treatment of a subject with a variant having a subset of the biological activities of the naturally occurring form of the reagent has fewer side effects in a subject relative to treatment with the naturally occurring form of the reagent.

Preferably, the analog, variant, or derivative reagent is functionally active. As utilized herein, the term “functionally active” refers to species displaying one or more known functional attributes of a full-length reagent. “Variant” refers to a reagent differing from naturally occurring reagent, but retaining essential properties thereof. Generally, variants are overall closely similar, and in many regions, identical to the naturally occurring reagent.

Variants of the reagent that function as either agonists (mimetics) or as antagonists can be identified by screening combinatorial libraries of mutants of the reagent for peptide agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential sequences is expressible as individual peptides, or alternatively, as a set of larger fusion proteins (e.g., for phage display) containing the set of sequences therein. There are a variety of methods which can be used to produce libraries of potential variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential sequences. Methods for synthesizing degenerate oligonucleotides are known in the art (see, e.g., Narang (1983) Tetrahedron 39:3; Itakura et al. (1984) Annu Rev Biochem 53:323; Itakura et al. (1984) Science 198:1056; Ike et al. (1983) Nucl. Acids Res. 11:477.

Derivatives and analogs of the reagent or individual moieties can be produced by various methods known within the art. For example, the polypeptide sequences may be modified by any number of methods known within the art. See e.g., Sambrook, et al., 1990. Molecular Cloning: A Laboratory Manual, 2nd ed., (Cold Spring Harbor Laboratory Press; Cold Spring Harbor, N.Y.). Modifications include: glycosylation, acetylation, phosphorylation, amidation, derivatization by known protecting/blocking groups, linkage to an antibody molecule or other cellular reagent, and the like. Any of the numerous chemical modification methodologies known within the art may be utilized including, but not limited to, specific chemical cleavage by cyanogen bromide, trypsin, chymotrypsin, papain, V8 protease, NaBH₄, acetylation, formylation, oxidation, reduction, metabolic synthesis in the presence of tunicamycin, etc.

Derivatives and analogs may be full length or other than full length, if said derivative or analog contains a modified nucleic acid or amino acid, as described infra. Derivatives or analogs of the reagent include, but are not limited to, molecules comprising regions that are substantially homologous in various embodiments, of at least 30%, 40%, 50%, 60%, 70%, 80%, 90% or preferably 95% amino acid identity when: (i) compared to an amino acid sequence of identical size; (ii) compared to an aligned sequence in that the alignment is done by a computer homology program known within the art (e.g., Wisconsin GCG software) or (iii) the encoding nucleic acid is capable of hybridizing to a sequence encoding the aforementioned peptides under stringent (preferred), moderately stringent, or non-stringent conditions. See, e.g., Ausubel, et al., Current Protocols in Molecular Biology, John Wiley and Sons, New York, N.Y., 1993.

Derivatives of the reagent may be produced by alteration of their sequences by substitutions, additions or deletions that result in functionally-equivalent molecules. One or more amino acid residues within the reagent may be substituted by another amino acid of a similar polarity and net charge, thus resulting in a silent alteration. Conservative substitutes for an amino acid within the sequence may be selected from other members of the class to which the amino acid belongs. For example, nonpolar (hydrophobic) amino acids include alanine, leucine, isoleucine, valine, proline, phenylalanine, tryptophan and methionine. Polar neutral amino acids include glycine, serine, threonine, cysteine, tyrosine, asparagine, and glutamine. Positively charged (basic) amino acids include arginine, lysine and histidine. Negatively charged (acidic) amino acids include aspartic acid and glutamic acid.

The reagent can be administered locally to any loci implicated in the CNS disorder pathology, e.g. any loci deficient in neural cells as a cause of the disease. For example, the reagent can be administered locally to the ventricle of the brain, substantia nigra, striatum, locus ceruleous, nucleus basalls of Meynert, pedunculopontine nucleus, cerebral cortex, spinal cord and retina.

Neural stem cells and their progeny can be induced to proliferate, differentiate, survive or migrate in vivo by administering to the host a reagent, alone or in combination with other agents, or by administering a pharmaceutical composition containing the reagent that will induce proliferation and differentiation of the cells. Pharmaceutical compositions include any substance that blocks the inhibitory influence and/or stimulates neural stem cells and stem cell progeny to proliferate, differentiate, migrate and/or survive. Such in vivo manipulation and modification of these cells allows cells lost, due to injury or disease, to be endogenously replaced, thus obviating the need for transplanting foreign cells into a patient.

Antibodies

Included in the invention are antibodies to be used as reagents. The term “antibody” as used herein refers to immunoglobulin molecules and immunologically active portions of immunoglobulin (Ig) molecules, e.g., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated protein of the invention intended to serve as an antigen, or a portion or fragment thereof, can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of Reelin, Gas6, Protein S, a molecule that regulates the phosphorylation status of Dab1 or a Reelin, Gas6 or Protein S receptor that is located on the surface of the protein, e.g., a hydrophilic region. A hydrophobicity analysis of the human those protein sequences will indicate which regions of the polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J Mol. Biol. 157: 105-142, each incorporated herein by reference in their entirety. Antibodies that are specific for one or more domains within an antigenic protein, or derivatives, fragments, analogs or homologs thereof, are also provided herein.

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. A Reelin, Gas6, Protein S, a molecule that regulates the phosphorylation status of Dab1 or ligand or receptor polypeptide or a fragment thereof comprises at least one antigenic epitope. An anti-Reelin, anti-Gas6 or anti-Protein A antibody of the present invention is said to specifically bind to the antigen when the equilibrium binding constant (K_{D}) is ≦1 μM, preferably ≦100 nM, more preferably ≦10 nM, and most preferably ≦100 pM to about 1 pM, as measured by assays such as radioligand binding assays or similar assays known to those skilled in the art.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). It is an objective, especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding,1986). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al, (Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994). Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA or TETA. Another bispecific antibody of interest binds the protein antigen described herein and further binds tissue factor (TF).

Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al ., J. Biol. Chem., 257: 286-288 (1982) via a disulfide-interchange reaction.

Antibody Therapeutics

Antibodies of the invention, including polyclonal, monoclonal, humanized and fully human antibodies, may used as therapeutic agents such as one of this invention. Such agents will generally be employed to treat or prevent a disease or pathology, specifically neurological disease, in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous Reelin, Gas6 or Protein S ligand to which it naturally binds. In this case, the antibody binds to the target and masks a binding site of the naturally occurring ligand, wherein the ligand serves as an effector molecule. Thus, the receptor mediates a signal transduction pathway for which ligand is responsible.

Alternatively, the effect may be one in which the antibody elicits a physiological result by virtue of binding to an effector binding site on the target molecule. In this case the target, a Reelin, Gas6 or Protein S receptor having an endogenous ligand which needs to be modulated, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen and the rate at which an administered antibody is depleted from the free volume of the subject to which it is administered.

Diseases and Disorders

Diseases and disorders that are characterized by altered (relative to a subject not suffering from the disease or disorder) levels or biological activity may be treated with therapeutics that antagonize (e.g., reduce or inhibit) or activate Reelin, Gas6 or Protein S activity. Therapeutics that antagonize activity may be administered in a therapeutic or prophylactic manner. Therapeutics that may be utilized include, but are not limited to: (i) an aforementioned peptide, analog, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (e.g., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (e.g., inhibitors, agonists and antagonists, including additional peptide mimetic of the invention or antibodies specific to a peptide of the invention) that alter the interaction between an aforementioned peptide and its binding partner.

Diseases and disorders that are characterized by altered (relative to a subject not suffering from the disease or disorder) levels or biological activity may be treated with therapeutics that increase (e.g., are agonists to) activity. In a preferred embodiment, the diseases to be treated include Alzheimer's disease, stroke, Parkinson's disease. Therapeutics that upregulate activity may be administered in a therapeutic or prophylactic manner. Therapeutics that may be utilized include, but are not limited to, an aforementioned peptide, analog, derivatives, fragments or homologs thereof, or an agonist that increases bioavailability.

Increased or decreased levels can be detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Therapeutic Methods

Another aspect of the invention pertains to methods of modulating Reelin, Gas6 or Protein S expression or activity for therapeutic purposes. The modulatory method of the invention involves contacting a cell with an agent that modulates one or more of the activities of Reelin, Gas6, Protein S or a molecule that regulates the phosphorylation status of Dab1 associated with the cell. An agent that modulates this protein activity can be an agent as described herein, such as a nucleic acid or a protein, a naturally-occurring cognate ligand of a Reelin, Gas6 or Protein S receptor, a peptide, a Reelin, Gas6 or Protein S peptidomimetic, or other small molecule. In one embodiment, the agent stimulates the activity of the Reelin, Gas6 or Protein S signaling pathway. Examples of such stimulatory agents include active Reelin, Gas6 or Protein S protein and a nucleic acid molecule encoding Reelin, Gas6 or Protein S that has been introduced into the cell. In another embodiment, the agent inhibits Reelin, Gas6 or Protein S signaling. Examples of such inhibitory agents include antisense nucleic acid molecules and antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder, specifically a neurological disorder. In one embodiment, the method involves administering a reagent (e.g., an reagent identified by a screening assay described herein), or combination of reagents that modulate (e.g., up-regulates or down-regulates) Reelin, Gas6, Protein S or Dab1 expression or activity. In another embodiment, the method involves administering a Reelin, Gas6, Protein S or a molecule that regulates the phosphorylation status of Dab1 protein or nucleic acid molecule as therapy to modulate proliferation, differentiation, migration and/or survival of NSCs/NPCs.

Stimulation of Reelin, Gas6, Protein S or Dab1 activity is desirable in situations in which Reelin, Gas6 and/or Protein S are abnormally downregulated and/or in which increased Reelin, Gas6 or Protein S activity is likely to have a beneficial effect. One example of such a situation is where a subject has a disorder characterized by aberrant cell proliferation and/or differentiation (e.g., Parkinson's disease and Alzheimer's disease).

Determination of the Biological Effect of the Therapeutic

In various embodiments of the invention, suitable in vitro or in vivo assays are performed to determine the effect of a specific therapeutic and whether its administration is indicated for treatment of the affected tissue.

In various specific embodiments, in vitro assays may be performed with representative stem cells or newly differentiated cells involved in the patient's disorder, to determine if a given therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Pharmaceutical Compositions

The invention provides methods of influencing central nervous system cells to produce progeny that can replace damaged or missing neurons in the central nervous system or other central nervous system cell types by exposing a patient, suffering from a neurological disease or disorder, to a reagent (e.g. Reelin, Gas6, Protein S) in a suitable formulation through a suitable route of administration, that modulates NSC or NPC activity in vivo. A “neurological disease or disorder” is a disease or disorder that results in the disturbance in the structure or function of the central nervous system resulting from developmental abnormality, disease, injury or toxin. Examples of neurological diseases or disorders include neurodegenerative disorders (e.g. associated with Parkinson's disease, Alzheimer's disease, Huntington's disease, Shy-Drager Syndrome, Progressive Supranuclear Palsy, Lewy Body Disease or Amyotrophic Lateral Sclerosis); ischemic disorders (e.g. cerebral or spinal cord infarction and ischemia, stroke); traumas (e.g. caused by physical injury or surgery, and compression injuries; affective disorders (e.g. stress, depression and post-traumatic depression); neuropsychiatric disorders (e.g. schizophrenia, multiple sclerosis or epilepsy); and learning and memory disorders.

This invention provides a method of treating a neurological disease or disorder comprising administering a reagent that modulates neural stem cell or neural progenitor cell activity in vivo to a mammal. The term “mammal” refers to any mammal classified as a mammal, including humans, cows, horses, dogs, sheep and cats. In one embodiment, the mammal is a human.

The invention provides a regenerative cure for neurodegenerative diseases by stimulating ependymal cells and subventricular zone cells to proliferate, migrate, differentiate and survive into the desired neural phenotype targeting loci where cells are damaged or missing. In vivo stimulation of ependymal stem cells is accomplished by locally administering a reagent to the cells in an appropriate formulation. By increasing neurogenesis, damaged or missing neurons can be replaced in order to enhance brain function in diseased states.

A pharmaceutical composition useful as a therapeutic agent for the treatment of central nervous system disorders is provided. For example, the composition includes a reagent of the invention, which can be administered alone or in combination with the systemic or local co-administration of one or more additional agents. Such agents include preservatives, ventricle wall permeability increasing factors, stem cell mitogens, survival factors, glial lineage preventing agents, anti-apoptotic agents, anti-stress medications, neuroprotectants, and anti-pyrogenics. The pharmaceutical composition preferentially treats CNS diseases by stimulating cells (e.g., ependymal cells and subventricular zone cells) to proliferate, migrate and differentiate into the desired neural phenotype, targeting loci where cells are damaged or missing.

A method for treating a subject suffering from a CNS disease or disorder is also provided. This method comprises administering to the subject an effective amount of a pharmaceutical composition containing a reagent (1) alone in a dosage range of 0.001 ng/kg/day to 10 mg/kg/day, preferably in a dosage range of 0.01 ng/kg/day to 5 mg/kg/day, more preferably in a dosage range of 0.1 ng/kg/day to 1 mg/kg/day, most preferably in a dosage range of 0.1 ng/kg/day to 1 μg/kg/day, (2) in a combination with a ventricle wall permeability increasing factor, or (3) in combination with a locally or systemically co-administered agent.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., chimeric peptide) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g. with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit form of the invention are dictated by and directly dependent on the unique characteristics of the active compound and the particular therapeutic effect to be achieved, and the limitations inherent in the art of compounding such an active compound for the treatment of individuals.

Nucleic acid molecules encoding a proteinaceous agent can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

In another embodiments, the reagent is administered in a composition comprising at least 90% pure reagent. The reagent can be, for example, Reelin, Gas6, Protein S or a Reelin, Gas6 or Protein S receptor, or any combination thereof.

Preferably the reagent is formulated in a medium providing maximum stability and the least formulation-related side-effects. In addition to the reagent, the composition of the invention will typically include one or more protein carrier, buffer, isotonic salt and stabilizer.

In some instances, the reagent can be administered by a surgical procedure implanting a catheter coupled to a pump device. The pump device can also be implanted or be extracorporally positioned. Administration of the reagent can be in intermittent pulses or as a continuous infusion. Devices for injection to discrete areas of the brain are known in the art (see, e.g., U.S. Pat. Nos. 6,042,579; 5,832,932; and 4,692,147).

Reagents containing compositions can be administered in any conventional form for administration of a protein. A reagent can be administered in any manner known in the art in which it may either pass through or by-pass the blood-brain barrier. Methods for allowing factors to pass through the blood-brain barrier include minimizing the size of the factor, providing hydrophobic factors which may pass through more easily, conjugating the protein reagent or other agent to a carrier molecule that has a substantial permeability coefficient across the blood brain barrier (see, e.g., U.S. Pat. No. 5,670,477).

Reagents, derivatives, and co-administered agents can be incorporated into pharmaceutical compositions suitable for administration. Such compositions typically comprise the agent and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions. Modifications can be made to the agents to affect solubility or clearance of the peptide. Peptidic molecules may also be synthesized with D-amino acids to increase resistance to enzymatic degradation. In some cases, the composition can be co-administered with one or more solubilizing agents, preservatives, and permeation enhancing agents.

For example, the composition can include a preservative or a carrier such as proteins, carbohydrates, and compounds to increase the density of the pharmaceutical composition. The composition can also include isotonic salts and redox-control agents.

In some embodiments, the composition administered includes the reagent and one or more agents that increase the permeability of the ventricle wall, e.g. “ventricle wall permeability enhancers.” Such a composition can help an injected composition penetrate deeper than the ventricle wall. Examples of suitable ventricle wall permeability enhancers include, for example, liposomes, VEGF (vascular endothelial growth factor), IL-s, TNFα, polyoxyethylene, polyoxyethylene ethers of fatty acids, sorbitan monooleate, sorbitan monolaurate, polyoxyethylene monolaurate, polyoxyethylene sorbitan monolaurate, fusidic acid and derivatives thereof, EDTA, disodium EDTA, cholic acid and derivatives, deoxycholic acid, glycocholic acid, glycodeoxycholic acid, taurocholic acid, taurodeoxycholic acid, sodium cholate, sodium glycocholate, glycocholate, sodium deoxycholate, sodium taurocholate, sodium glycodeoxycholate, sodium taurodeoxycholate, chenodeoxycholic acid, urosdeoxycholic acid, saponins, glycyrrhizic acid, ammonium glycyrrhizide, decamethonium, decamethonium bromide, dodecyltrimethylammonium bromide, and dimethyl-β-cyclodextrin or other cyclodextrins.

Drug Screening

The invention also provides a method of using the receptors or receptor/reagent complexes for analyzing or purifying certain stem or progenitor cell populations, using e.g. antibodies, against the receptors or receptor/reagent complexes.

In another aspect, the invention provides a method for screening for reagents that influence stem and progenitor cells. In some applications, neural cells (undifferentiated or differentiated) are used to screen factors that promote maturation into neural cells, or promote proliferation and maintenance of such cells in long-term culture. For example, candidate reagents are tested by adding them to cells in culture at varying dosages, and then determining any changes that result, according to desirable criteria for further culture and use of the cells. Physical characteristics of the cells can be analyzed by observing cell and neurite growth with microscopy. The induction of expression of increased levels of proliferation, differentiation and migration can be analyzed with any technique known in the art which can identify proliferation and differentiation. Such techniques include RT-PCR, in situ hybridization, and ELISA.

In one aspect, novel receptor/reagents in undifferentiated neurospheres was examined using RT-PCR techniques. In particular, genes that are up-regulated in these undifferentiated neurospheres were identified. As used herein, the term “up-regulation” refers to a process that increases reagent/receptor interactions due to an increase in the number of available receptors. The presence of these genes suggests a potential role in the mediation of signal transduction pathways in the regulation of NSC/NPC function. Furthermore, by knowing the levels of expression of the receptors or their various reagents, it is possible to diagnose disease or determine the role of stem and progenitor cells in the disease. By analyzing the genetic or amino-acid sequence variations in these genes or gene products, it is possible to diagnose or predict the development of certain diseases. Such analysis will provide the necessary information to determine the usefulness of using stem or progenitor cell based treatments for disease.

In another aspect, in situ hybridization is performed on adult mouse brain sections to determine which cells in the adult brain express these signaling pathways. This data is helpful in determining treatment options for various neurological diseases.

To determine the effect of a potential reagent on neural cells, a culture of NSCs/NPCs derived from multipotent stem cells can be obtained from normal neural tissue or, alternatively, from a host afflicted with a CNS disease or disorder. The choice of culture will depend upon the particular agent being tested and the effects one wishes to achieve. Once the cells are obtained from the desired donor tissue, they are proliferated in vitro in the presence of a proliferation-inducing reagent.

The ability of various biological agents to increase, decrease or modify in some other way the number and nature of the stem cell progeny proliferated in the presence of the proliferative factor can be screened on cells proliferated by the methods previously discussed. For example, it is possible to screen for reagents that increase or decrease the proliferative ability of NSCs/NPCs which would be useful for generating large numbers of cells for transplantable purposes. In these studies precursor cells are plated in the presence of the reagent in question and assayed for the degree of proliferation and survival or progenitor cells and their progeny can be determined. It is possible to screen neural cells which have already been induced to differentiate prior to the screening. It is also possible to determine the effects of the reagent on the differentiation process by applying them to precursors cells prior to differentiation. Generally, the reagent will be solubilized and added to the culture medium at varying concentrations to determine the effect of the agent at each dose. The culture medium may be replenished with the reagent every couple of days in amounts so as to keep the concentration of the reagent somewhat constant.

Changes in proliferation are observed by an increase or decrease in the number of neurospheres that form and/or an increase or decrease in the size of the neurospheres, which is a reflection of the rate of proliferation and is determined by the numbers of precursor cells per neurosphere.

Using these screening methods, it is possible to screen for potential drug side-effects on prenatal and postnatal CNS cells by testing for the effects of the biological agents on stem cell and progenitor cell proliferation and on progenitor cell differentiation or the survival and function of differentiated CNS cells.

Other screening applications of this invention relate to the testing of pharmaceutical compounds for their effect on neural tissue. Screening may be done either because the compound is designed to have a pharmacological effect on neural cells, or because a compound designed to have effects elsewhere may have unintended side effects on the nervous system. The screening can be conducted using any of the neural precursor cells or terminally differentiated cells of the invention.

Effect of cell function can be assessed using any standard assay to observe phenotype or activity of neural cells, such as receptor binding, proliferation, differentiation, survival-either in cell culture or in an appropriate model.

Therapeutic Uses

The fact that neural stem cells are located in the tissues lining ventricles of mature brains offers several advantages for the modification and manipulation of these cells in vivo and the ultimate treatment of various neurological diseases, disorders, and injury that affect different regions of the CNS. Therapy for these diseases can be tailored accordingly so that stem cells surrounding ventricles near the affected region would be manipulated or modified in vivo using the methods described herein. The ventricular system is found in nearly all brain regions and thus allows easier access to the affected areas. In order to modify the stem cells in vivo by exposing them to a composition comprising a reagent, it is relatively easy to implant a device that administers the composition to the ventricle and thus, to the neural stem cells. For example, a cannula attached to an osmotic pump may be used to deliver the composition. Alternatively, the composition may be injected directly into the ventricles. The neural stem cell progeny can migrate into regions that have been damaged as a result of injury or disease. Furthermore, the close proximity of the ventricles to many brain regions would allow for the diffusion of a secreted neurological agent by the stem cells or their progeny.

In an additional embodiment, a reagent of the invention is administered locally, as described above, in combination with an agent administered locally or systemically. Such agents include, for example, one or more stem cell mitogens, survival factors, glial-lineage preventing agents, anti-apoptotic agents, anti-stress medications, neuroprotectants, and anti-pyrogenics, or any combination thereof.

The agent is administered systemically before, during, or after administration of the reagent of the invention. The locally administered agent can be administered before, during, or after the reagent administration.

For treatment of Huntington's Disease, Alzheimer's Disease, Parkinson's Disease, and other neurological disorders affecting primarily the forebrain, a reagent alone or with an additional agent or agents is delivered to the ventricles of the forebrain to affect in vivo modification or manipulation of the stem cells. For example, Parkinson's Disease is the result of low levels of dopamine in the brain, particularly the striatum. It is therefore advantageous to induce a patient's own quiescent stem cells to begin to divide in vivo and to induce the progeny of these cells to differentiate into dopaminergic cells in the affected region of the striatum, thus locally raising the levels of dopamine.

Normally the cell bodies of dopaminergic neurons are located in the substantia nigra and adjacent regions of the mesencephalon, with the axons projecting to the striatum. The methods and compositions of the invention provide an alternative to the use of drugs and the controversial use of large quantities of embryonic tissue for treatment of Parkinson's disease. Dopamine cells can be generated in the striatum by the administration of a composition comprising a reagent of the invention to the lateral ventricle.

For the treatment of MS and other demyelinating or hypomyelinating disorders, and for the treatment of Amyotrophic Lateral Sclerosis or other motor neuron diseases, a reagent of the invention, alone or with an additional agent or agents is delivered to the central canal.

In addition to treating CNS tissue immediately surrounding a ventricle, a reagent of the invention, alone or with an additional agent or agents can be administered to the lumbar cistern for circulation throughout the CNS.

In other aspects, neuroprotectants can also be co-administered systemically or locally before, during and/or after infusion of a regent of the invention. Neuroprotectants include antioxidants (agents with reducing activity, e.g., selenium, vitamin E, vitamin C, glutathione, cysteine, flavinoids, quinolines, enzymes with reducing activity, etc), Ca-channel modulators, Na-channel modulators, glutamate receptor modulators, serotonin receptor agonists, phospholipids, unsaturated- and polyunsaturated fatty acids, estrogens and selective estrogen receptor modulators (SERMS), progestins, thyroid hormone and thyroid hormone-mimicking compounds, cyclosporin A and derivatives, thalidomide and derivatives, methylxanthines, MAO inhibitors; serotonin-, noradrenaline and dopamine uptake blockers; dopamine agonists, L-DOPA, nicotine and derivatives, and NO synthase modulators.

Certain reagents of the invention may be pyrogenic following IV injection (in rats; Am. J. Physiol. Regul. Integr. Comp. Physiol. 2000 278: R1275-81). Thus, in some aspects of the invention, antipyrogenic agents like cox2 inhibitors, indomethacin, salisylic acid derivatives and other general anti-inflammatory/anti-pyrogenic compounds can be systemically or locally administered before, during and/or after administration of the reagent of the invention.

In another aspect of the invention, anti-apoptotic agents including caspase inhibitors and agents useful for antisense-modulation of apoptotic enzymes and factors can be administered before, during, or after administration of the reagent of the invention.

Stress syndromes lower neurogenesis, therefore in some aspects, it may be desirable to treat a subject with anti-stress medications such as, e.g., anti-glucocorticoids (e.g., RU486) and beta-blockers, administered systemically or locally before, during and/or after infusion of the reagent of the invention.

Methods for preparing the reagent dosage forms are known, or will be apparent, to those skilled in this art.

The amount of reagent to be administered will depend upon the exact size and condition of the patient, but will be from 0.5 ng/kg/day to 500 ng/kg/day in a volume of 0.001 to 10 ml.

The duration of treatment and time period of administration of reagent will also vary according to the size and condition of the patient, the severity of the illness and the specific composition and method being used.

The effectiveness of each of the foregoing methods for treating a patient with a CNS disease or disorder is assessed by any known standardized test for evaluating the disease.

Other features of the invention will become apparent in the course of the following description of exemplary embodiments which are given for illustration of the invention and are not intended to be limiting thereof. All references, patents and patent applications cited are hereby incorporated by reference in their entirety.

### EXAMPLES

### Example 1

### Expression of Dab1, Reelin and its receptors VLDLR2, ApoER2

Methods

A. Mouse & Human Cultures

Mouse Neurosphere Cultures

The anterior lateral wall of the lateral ventricle of 5-6 week old mice was enzymatically dissociated in 0.8 mg/ml hyaluronidase and 0.5 mg/ml trypsin in DMEM containing 4.5 mg/ml glucose and 80 units/ml DNase at 37° C. for 20 min. The cells were gently triturated and mixed with three volumes of Neurosphere medium (DMEM/F12, B27 supplement, 12.5 mM HEPES pH7.4) containing 20 ng/ml EGF (unless otherwise stated), 100 units/ml penicillin and 100 μg/ml streptomycin. After passing through a 70 μm strainer, the cells were pelleted at 160×g for 5 min. The supernatant was subsequently removed and the cells resuspended in Neurosphere medium supplemented as above, plated out in culture dishes and incubated at 37° C. Neurospheres were ready to be split approximately 7 days after plating.

To split neurosphere cultures, neurospheres were collected by centrifugation at 160×g for 5 min. The neurospheres were resuspended in 0.5 ml Trypsin/EDTA in HBSS (1×), incubated at 37° C. for 2 min and triturated gently to aid dissociation. Following a further 3 min incubation at 37° C. and trituration, 3 volumes of ice cold NSPH-media-EGF were added to stop further trypsin activity. The cells were pelleted at 220×g for 4 min, resuspended in fresh Neurosphere medium supplemented with 20 ng/ml EGF and I nM bFGF plated out and incubated at 37° C.

Human Neural Stem Cell (HNSC) Cultures

A biopsy from the anterior lateral wall of the lateral ventricle was taken from an adult human patient and enzymatically dissociated in PDD (Papain 2.5 U/ml; Dispase 1 U/ml; Dnase I 250 U/ml) in DMEM containing 4.5 mg/ml glucose and 37° C. for 20 min. The cells were gently triturated and mixed with three volumes of Human Neural Stem Cell Plating Medium (HNSCPM) (DMEM/F12; 10% fetal bovine serum (FBS)). The cells were pelleted at 250×g for 5 min. The supernatant was subsequently removed and the cells resuspended in HNSCPM, plated out on fibronectin coated culture dishes and incubated at 37° C. in 5% CO₂. The following day the expansion of the culture was initiated by change of media to UNSC culture media (DMEM/F12; BIT 9500; EGF 20 ng/ml; FGF2 20 ng/ml). The HNSC were split using trypsin and EDTA under standard conditions. FBS was subsequently added to inhibit the reaction and the cells collected by centrifugation at 250×g for 5 min. The HNSC were replated in HNSC culture media.

B. RT-PCR

Mouse Neurospheres

The following primer pairs were designed to specifically identify the presence of reelin, vldlr, apoER2 and dab1 gene expression in neurospheres. Estimated band sizes for each primer pair are given below:

Band size
(base pairs)

| | | |
|---|---|---|
| reelin | AGAACTGGAGGCGGGTCACG | 524 |
| | (SEQ ID NO:1) | |
| | TTCATCCATCAGGTGCCAGGTG | |
| | (SEQ ID NO:2) | |
| | TGTGAATATCCTTCTCCCTCCTG | 392 |
| | (SEQ ID NO:3) | |
| | AGTTCTCAGTGGGCGTCAGGTC | |
| | (SEQ ID NO:4) | |
| vldlr | GGAAAGTTCAAGTGCAGAAGCG | 1061 |
| | (SEQ ID NO:5) | |
| | CCAGTCAATTTATTGGCACCG | |
| | (SEQ ID NO:6) | |
| | GGAAAGTTCAAGTGCAGAAGCG | 370 |
| | (SEQ ID NO:7) | |
| | AAGATCAGACTCGGCTCTTTGC | |
| | (SEQ ID NO:8) | |
| apoER2 | TTCAGTGTAAGAGTGGCGAGTGC | 519 |
| | (SEQ ID NO:9) | |
| | CGCTGTAGATCTTGCGGTAGG | |
| | (SEQ ID NO:10) | |
| | TTCAGTGTAAGAGTGGCGAGTGC | 401 |
| | (SEQ ID NO:11) | |
| | CCAGGTCTATCCTCCGCACC | |
| | (SEQ ID NO:12) | |
| dab1 | AGGATCCTCGATGAGCCTGG | 760 |
| | (SEQ ID NO:13) | |
| | TAATTCTTCTGCAGGTTCTGC | |
| | (SEQ ID NO:14) | |
| | AGGATCCTCGATGAGCCTGG | 355 |
| | (SEQ ID NO:15) | |
| | AAAGATTTTGATTCCTCCAAAGG | |
| | (SEQ ID NO:16) | |

Neurospheres were prepared from the LVW as stated above. 3 days after the first split, the neurospheres were harvested and total RNA isolated using Qiagen's RNeasy Mini Kit according to the manufacturer's instructions. Life Technology's One-Step RT-PCR Kit was used to detect the presence of reelin, vldlr, apoER2 and dab1 mRNA. Briefly, 12.5 ng of total RNA was used in each reaction, with an annealing temperature of 55° C. or 60° C. To ensure that genomic contamination of the total RNA did not give rise to false positive results, an identical reaction in which the RT-taq polymerase mix was replaced by taq polymerase alone and was run in parallel with the experimental RT-PCR. The reactions were electrophoresed on a 1.5% agarose gel containing ethidium bromide and the bands visualized under UV light. Bands corresponding to the estimated length of PCR products of the desired genes were cloned into the cloning vector pGEM-Teasy and sequenced to verify their identity.

Human Neural Stem Cells

The following primer pairs were designed to specifically identify the presence of vldlr, apoer2, reelin and dab1 gene expression in HNSC cultures. Estimated band sizes for each primer pair are given below:

Band size
(base pairs)

| | | |
|---|---|---|
| vldlr | CCACCGGAACCGGGAGAAAA | 234 |
| | (SEQ ID NO:17) | |
| | GCGCTTTCATCTGAACCATCTTCG | |
| | (SEQ ID NO:18) | |
| | CCACCGGAACCGGGAGAAAA | 296 |
| | (SEQ ID NO:19) | |
| | GGCGCCACAGCTGATTTCATG | |
| | (SEQ ID NO:20) | |
| apoer2 | CCGGCCAAGGAGTGCGAAAA | 241 |
| | (SEQ ID NO:21) | |
| | TGCAAGTGGCCTCGGACTCATC | |
| | (SEQ ID NO:22) | |
| | CCGGCCAAGGAGTGCGAAAA | 371 |
| | (SEQ ID NO:23) | |
| | AAGGTAGCACAGCCGGCCTCAT | |
| | (SEQ ID NO:24) | |
| reelin | CCAGTTTATGTGCAGTGTGGTAGCC | 274 |
| | (SEQ ID NO:25) | |
| | TTGCAGTTGGTGGTAGGAGTCAAAG | |
| | (SEQ ID NO:26) | |
| | CCAGTTTATGTGCAGTGTGGTAGCC | 349 |
| | (SEQ ID NO:27) | |
| | ATTGCCATGCATAATCGCGC | |
| | (SEQ ID NO:28) | |
| dab1 | AAGAGGTTTAAAGGTGAAGGGGTCC | 275 |
| | (SEQ ID NO:29) | |
| | CGGTGATCTGTAATGTCCTTTGCAA | |
| | (SEQ ID NO:30) | |
| | AAGAGGTTTAAAGGTGAAGGGGTCC | 316 |
| | (SEQ ID NO:31) | |
| | ATCTGTGATTCCCTTCCTTCCCAC | |
| | (SEQ ID NO:32) | |

HNSC were prepared and cultured as stated above. Total RNA isolated using Qiagen's RNeasy Mini Kit according to the manufacturer's instructions and DNase treated using Ambion DNase I and according to protocol. Life Technology's One-Step RT-PCR Kit was used to detect the presence of vldlr, apoer2, reelin, and dab1 mRNA. Briefly, 50 ng of total RNA was used in each reaction, with an annealing temperature of 55° C. To further ensure that genomic contamination of the total RNA did not give rise to false positive results, an identical reaction in which the RT-taq polymerase mix was replaced by Taq polymerase alone and was run in parallel with the experimental RT-PCR. The reactions were electrophoresed on a 1.5% agarose gel containing ethidium bromide and the bands visualised under UV light. Bands corresponding to the estimated length of PCR products of the desired genes were cloned into the cloning vector pGEM-Teasy and sequenced to verify their identity.

C. In Situ Hybridization

Preparation of Tissue

Whole brain from 6 weeks old mice were dissected out and frozen at −80° C. 12 μm sections were cut in a cryostat and lifted from the knife with room temperature Superfrost Plus slides (Merck). The slides with sections were placed on a slide warmer at 42° C. briefly to dry, placed in 3.7% formaldehyde solution in 1×phosphate buffered saline (PBS) and rinsed twice with 1 ×PBS. The sections were acetylated in 0.25% acetic anhydride in 0.1 M triethanolamine solution, pH 8.0, for 20 min and dehydrated and in a series of increasing EtOH concentrations; 70% ethanol for 1 min., 80% ethanol for 1 min., 95% ethanol for 2 min., 100% ethanol for 1 min., chloroform for 5 min. to remove lipids, 100% ethanol for 1 min., and 95% ethanol for 1 min.

Hybridization

A 751 bp antisense riboprobe (cRNA probe) corresponding to bases 963 to 1713 of the coding sequence of murine vldlr gene was transcribed in vitro and concurrently digoxigenin-11-UTP-labeled using a commercially available kit (Roche) according to the manufacturer's instruction. The slides were placed in a humidified histochemistry chamber (Merck) and a hybridization solution containing 51% formamide; 10% dextran sulfate; 10 mM Tris-HCl, pH 8.0; 1 mM EDTA, pH 8.0; 205 mM NaCl; 1×Denhardt's solution; 50 ug/ml tRNA; 10 mM dithiothreitol and 300 ng/ml labeled probe was placed on top of the sections and covered with Parafilm and allowed to hybridize for 20 hrs at 55° C. The following day the slides were rinse 4 times 5 min. in 4×saline sodium citrate (SSC) at room temperature and incubated in RNase solution (10 ng/ml Rnase A; 500 mM NaCl; 100 mM Tris-HCl, pH 8.0; 10 mM EDTA) at 37° C. for 30 min to digest unhybridized probe. The sections were then rinsed twice for 5 min in 2×SSC followed by a 10 min rinse in 1×SSC and 10 min in 0.5×SSC at room temperature. Next, the slides were washed at 70° C. in 0.1×SSC followed by two 5-min washes in room temperature 1×SSC.

Detection of Digoxigenin-labeled Probes

The slides were transferred from the SSC wash above to Buffer 1 (100 mM Tris-HCl, 150 mM NaCl, pH7.5) for two 5-min washes. The sections were blocked in 5% normal goat serum (NGS; Sigma) and 0.1% Triton X-100 for 30 min. The slides were then transferred to Buffer 1 with 1% NGS, 0.1% Triton X-100, and 1:200 sheep polyclonal anti-digoxigenin-AP (Roche) for 2 hrs at room temperature. The antibody was then washed away in Buffer 1 twice for 10 min. Prior to detection, slides were incubated in Buffer 2 (100 mM Tris-HCl, 100 mM NaCl, 50 mM MgCl₂, pH9.5) for 5 min. Detection was carried out for several hours to overnight at room temperature in the dark in Buffer 3 with 0.34 mg/ml nitroblue tetrazolium chloride and 0.18 mg/ml 5-bromo-4-chloro-3-indolyl phosphate p-toluidinium salt, 10% polyvinyl alcohol, and levamisole (240 μg/ml, to block peripheral-type endogenous alkaline phosphatase). When the staining was determined optimal, the detection was stopped in TE buffer for 30 min. The sections were dipped in 95% EtOH 2×15 s and dipped briefly in distilled water. Sections were counterstained in 0.1% FCF Green and mounted using Mowiol.

As negative controls, two slides were treated with RNase (10 ng/ml Rnase A; 500 mM NaCl; 100 mM Tris-HCl, pH 8.0; 10 mM EDTA) at 37° C. for 30 min before included in the hybridizations. Some staining remained in the dentate gyrus of the hippocampus, but otherwise only background levels could be detected.

D. Radioactive in Situ Hybridization

Sections (14 82 m) of whole mouse brain were cut on a cryostat at −17° C., thawed onto microscope slides (Superfrost Plus, BDH, UK) and fixed in 4% formaldehyde for 5 min, deproteinated for 15 min in 0.2 M HCl, treated in 0.25% acetic anhydride in 0.1 M triethanolamine buffer, pH 8.0 for 20 min and dehydrated in an ascending series of ethanol concentrations including a 5 min chloroform step prior to hybridization. To detect reelin and dab1 mRNA, antisense cRNA probes were transcribed from plasmids (pGEM-Teasy) containing reelin cDNA (392 base pairs corresponding to bases 7869 to 8260 of the coding sequence of the Reelin gene) or dab1 cDNA (337 base pairs corresponding to 64 bases of the 5′ UTR to base number 273 of the coding sequence of the dab1 gene) and concurrently [α-³⁵S]UTP-labeled. The sections were incubated with the probe at 55° C. for 16 h in a hybridization buffer containing 52% formamide, 10% Dextran Sulfate, 208 mM NaCl, 2% 50×Denhardt's solution (1% Ficoll, 1% polyvinylpyrrolidene, 1% BSA) 10 mM Tris pH 8.0, 1 mM EDTA, 500 ng/ml yeast tRNA, 10 mM dithiothreitol (DTT) and 20×10⁶ cpm probe per ml buffer. After hybridization, the sections were treated with RNase A, 10 μg/ml in 0.5 M NaCl, at 37° C. for 30 min and washed in 4×saline sodium citrate (SSC; 1×SSC is 0.15 M sodium chloride, 0.015 M trisodium citrate pH 7.0) for 20 min, 2×SSC for 10 min., 1×SSC for 10 min. and 0.5×SSC for 10 min. at room temperature. A high stringency wash was carried out at 70° C. for 30 min in 0.1×SSC. All wash steps included the addition of 1 mM DTT. The sections were dehydrated in a ascending series of ethanol concentrations, dried over night and mounted in cassettes with autoradiographic films (Beta-max, Amersham) layed on top for 3 weeks. The films were developed in Kodak D-19 developer, fixed in Kodak RA-3000 diluted 1:3, rinsed and dried. The sections were then dipped in Kodak NTB-2 nuclear track emulsion diluted 1:1, exposed for six weeks, developed in Kodak D-19 for 3 min., fixed in Kodak RA-3000 fixer and counterstained with cresyl violet. The specificity of the hybridization was tested using a sense probe transcribed from the same plasmid. No hybridization signal was obtained under this condition. The emulsion dipped sections were analysed manually using a Nikon E600 microscope.

VLDLR, ApoER2, Reelin and Dab1 Gene Expression

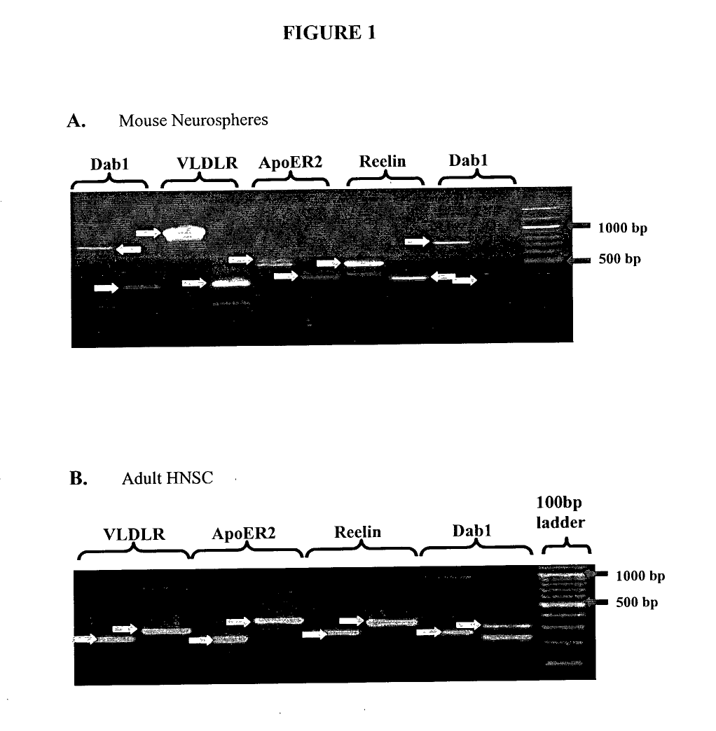

RT-PCR was performed on total RNA prepared from cultured non-adherent mouse neurospheres (FIG. 1A) and adult HNSC (FIG. 1B) using primer pairs specific for the mouse and human genes above. The bands indicated with a arrows correspond to the bands of the desired size. FIG 1A: Mouse Neurospheres (dab1 [lane1 760 bp; lane2 355 bp], vldlr [lane3 1061 bp; lane4 370 bp], apoer2 [lane5 519 bp; lane6 401 bp], reelin [lane7 524 bp; lane8 392 bp]). FIG. 1B: adult HNSC (vldlr [lane1 234 bp; lane2 296 bp], apoer2 [lane3 241 bp; lane4 371 bp], reelin [lane5 274 bp; lane6 349 bp], dab1 [lane7 275 bp; lane5 316 bp]). Sequencing of these bands confirmed that they represent correct product. A parallel control experiment without using any reverse transcriptase, only taq polymerase, ruled out false positive bands through genomic contamination (data not shown).

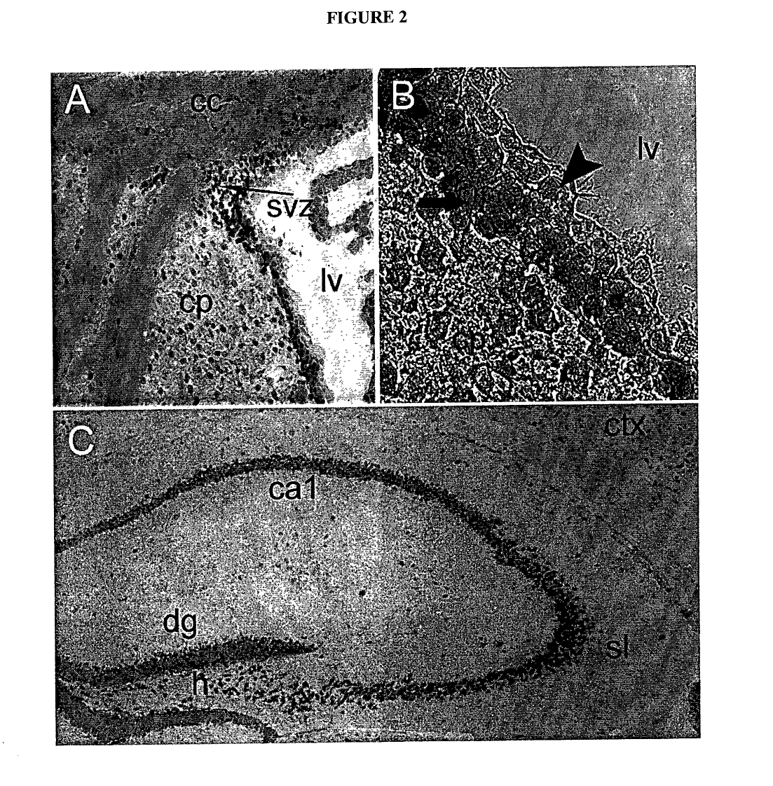

FIG. 2 represents brightfield micrographs of VLDLR mRNA positive cells in coronal sections of adult mouse brain. FIG. 2A shows VLDLR expression in the posterior lateral aspect of the lateral ventricle wall. Note the staining in the subventricular zone as indicated by the arrow. FIG. 2B shows a higher magnification of the lateral ventricle wall. FIG. 2C shows expression in hippocampus. Note high levels in the CA1 pyramidal layer and in stratum lucidem. A few scattered cells are seen in the dentate gyrus as well as throughout the layers surrounding the pyramidal cells. Abbreviations: cal, cornu ammoni field 1 of the hippocampus; cc, corpus callosum; cp, caudate putamen; ctx, cerebral cortex; dg, dentate gyrus of the hippocampus; h, hilus of the hippocampus; lv, posterior lateral ventricle; sl, stratum lucidem.

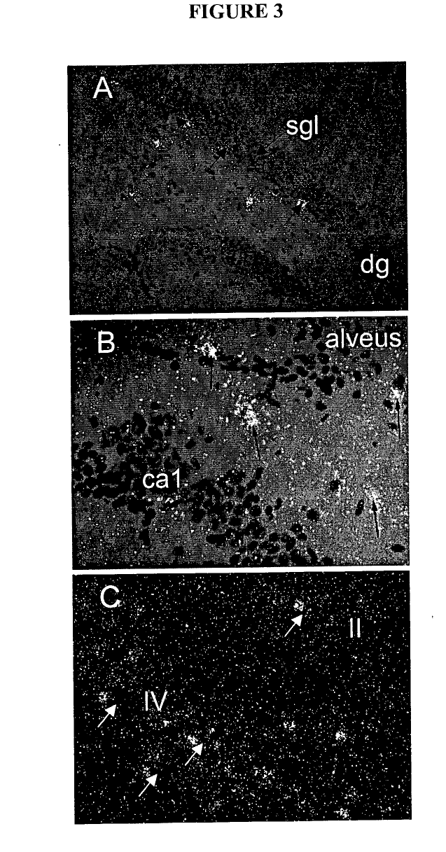

FIG. 3 illustrates darkfield microphotographs of reelin mRNA in coronal sections of adult mouse brain. FIG. 2A represents expression of reelin mRNA in the dentate gyrus of the hippocampus. Note the staining in the subgranular layer as indicated by the arrows. FIG. 3B represents a high magnification of the alveus and CA1 region of the hippocampus. Note the reelin positive staining of cells in the alveus and between the alveus and CA1 pyramidal layer, as indicated by the arrows. FIG. 3C show reelin mRNA expression in cortex. It is important to note the scattered positively labeled cells predominate in cortical layers II and IV, as indicated by arrows. Abbreviations: ca1, cornu ammoni field 1 of the hippocampus; dg, dentate gyrus of the hippocampus; sg1, subgranular layer of dentate gyrus; II, layer II of the cerebral cortex; IV, layer IV of the cerebral cortex.

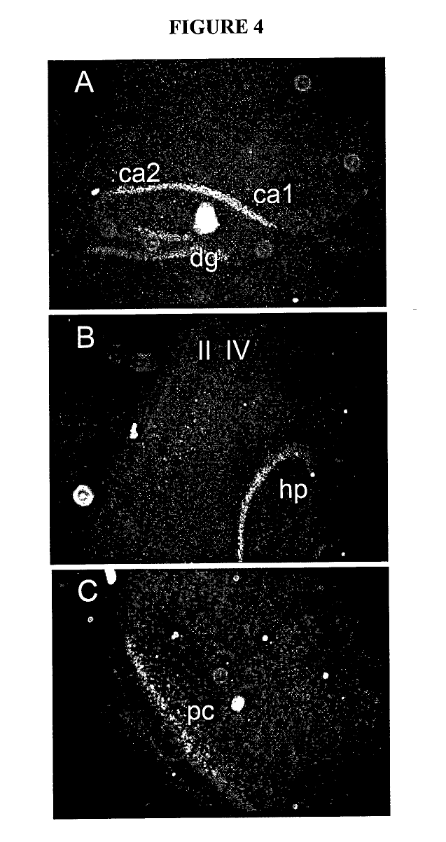

FIG. 4 illustrates darkfield microphotographs of dab1 mRNA in coronal sections of adult mouse brain. FIG. 4A represents dab1 expression in the hippocampus. Note high levels in the CA1 and CA2 pyramidal layers as well as throughout dentate gyrus. FIG. 4B represents dab1 expression in cortex. Note the scattered positively labeled cells predominate in cortical layers II and IV. FIG. 4C represents the expression in piriform cortex. Abbreviations: ca1 , cornu ammoni field 1 of the hippocampus; ca2, cornu ammoni field 1 of the hippocampus; dg, dentate gyrus of the hippocampus; hp, hippocampus; pc, piriform cortex.

### Example 2

### In Vivo Analysis of Reelin on Neural Proliferation

Methods

A. In Vivo Cell Proliferation Labeling

BrdU (1 mg/ml) was supplemented to the drinking water of adult (six weeks old) mice (4 reeler and 4 wild-type) for two weeks. After the allotted time the mice were sacrificed and the brains removed and frozen at −70° C. prior to sectioning for immumohistochemical analysis.

B. Immunohistochemistry of the Mouse Brain

Brains were cut into 20-μm coronal sections using a cryostat-microtome. The sections were thawed onto pretreated slides and fixed in 4% (wt/vol) paraformaldehyde/PBS for 10 min. After washing in PBS, the sections were treated with 2M HCl at 37° C. for 30 min to increase accessibility of the anti-BrdU antibody to the cell nuclei. The sections were rinsed in PBS and transferred to blocking solution (PBS; 0.1% Tween; 10% goat serum) overnight at 4° C. Primary antibody (rat anti-BrdU, Harlan Sera Labs) was applied at 1:100 in blocking solution for 90 min at room temperature. After washing in PBS/0.1% Tween for 3×30 min, secondary biotinylated antibody (goat anti-rat, VectorLabs) was added at a 1:200 dilution in blocking solution for 60 min at room temperature. The sections were washed for 2 hours prior to treatment with Vectastain Kit (VectorLabs) according to the manufacturer's protocol. After 1 hour of washing, the BrdU-antibody complex was detected using 0.05% diaminobenzidine with 0.01% H₂O₂, and counterstained with Hematoxylin. The sections were dehydrated in a graded series of ethanol concentrations, followed by xylene and 99% ethanol, and mounted in Pertex. Sections were visualised using a Nikon Eclipse E600 microscope and pictures taken with a Spot Insight CCD camera.

The procedure for doubling labeling of BrdU with glial fibrillary acidic protein (GFAP) or Doublecortin (DTX) was performed sequentially. Briefly, following fixation in 4% (wt/vol) paraformaldehyde/PBS for 10 min, the sections were incubated in overnight blocking solution ([GFAP blocking solution, PBS; 0.1% Tween; 10% goat serum] [DTX blocking solution, PBS; 0.1% Tween; 10% rabbit serum]) at 4° C. Anti-GFAP (rabbit polyclonal antibody, DAKO) and anti-DTX (goat polyclonal antibody, Santa Cruz) were applied at 1:400 and 1:100 respectively in their appropriate blocking solution for 60 min at room temperature. After washing in PBS/0.1% Tween for 3×30 min, secondary FITC-conjugated antibody ([GFAP, goat anti-rabbit, Vector Laboratories, CA] [DTX, rabbit anti-goat, Vector Laboratories, CA]) was added at a 1:200 dilution in their appropriate blocking solution for 60 min at room temperature. The sections were washed for 2 hours prior to post-fixation in 4% (wt/vol) paraformaldehyde /PBS for 10 min followed by treatment with 2M HCl at 37° C. for 30 min to increase accessibility of the anti-BrdU antibody to the cell nuclei. The sections were rinsed in PBS and transferred to blocking solution (PBS; 0.1% Tween; 10% goat serum) overnight at 4° C. Primary antibody (rat anti-BrdU, Harlan Sera Labs) was applied at 1:100 in blocking solution for 90 min at room temperature. After washing in PBS/0.1% Tween for 3×30 min, secondary Texas Red-conjugated antibody (goat anti-rat, VectorLabs) was added at a 1:200 dilution in blocking solution for 60 min at room temperature. The sections were washed for 2 hours prior to mounting onto glass slides. Sections were visualised using a Nikon Eclipse E600 microscope and pictures taken with a Spot Insight CCD camera.

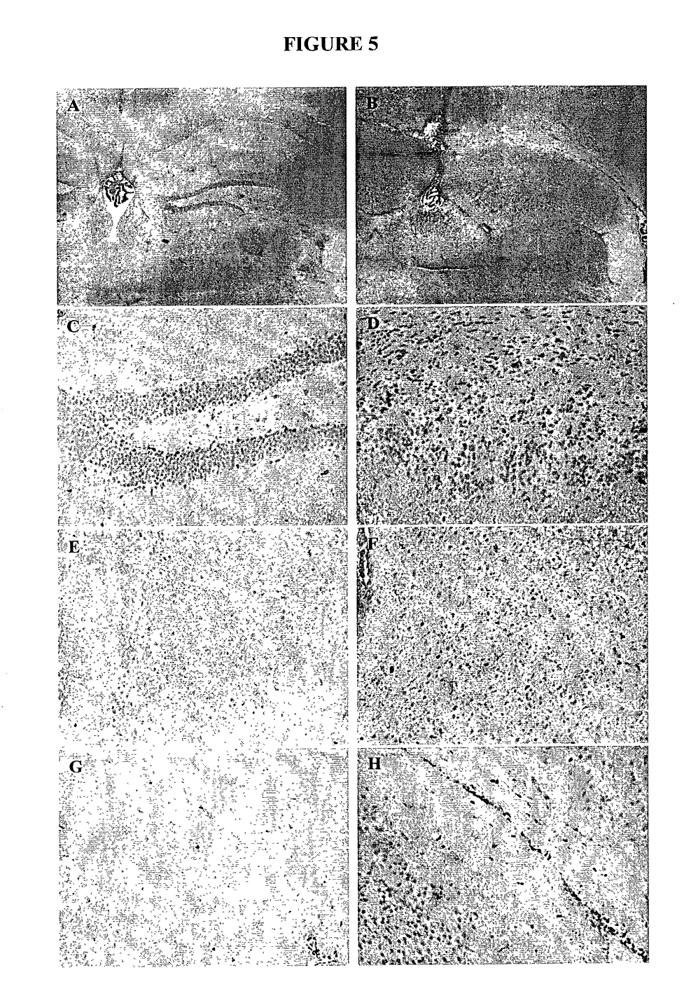

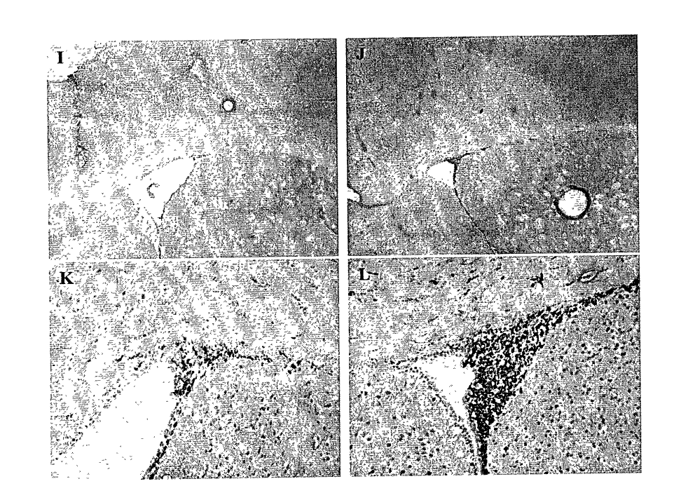

FIG. 5 represents immunohistochemistry of wild-type and Reeler brains after two weeks of BrdU treatment. BrdU incorporation into the nuclei of dividing cells is a standard method of labeling proliferating populations of cells, and has become a well used marker for dividing stem cells and their progeny (Zhang, Zhang et al. 2001).

In this study, the endogenous neural proliferation in wild-type mice were compared to the Reeler mutant mouse using the BrdU incorporation detection method. Large scale disruptions are apparent in many structures of Reeler mice, in particular the cerebral cortex, where neuronal layering is inverted, the hippocampus, which is completely disorganized with no apparent CA1-3 and dentate gyrus formations, and the cerebellum. Overall there was significantly increased proliferation in the brain of Reeler mice (FIG. 5, black dots). Comparisons of specific regions of the brain showed a greater number of BrdU positive cells in the cerebral cortex, the hippocampus, alveus and most significantly the anterior subventricular zone of the lateral ventricle.

BrdU incorporated into the nuclei of proliferating cells was detected by DAB-immunohistochemistry, and all of the nuclei were counterstained with hematoxylin. The hippocampus of wild-type mice at low and high magnification respectively (FIGS. 5A and 5C) and corresponding sections of Reeler mice (FIGS. 5B and 5D) illustrate the disorganized nature of the hippocampus and slightly increased hippocampal proliferation in these animals. FIGS. 5E and 5F show the cerebral cortices of wild-type and Reeler respectively. Cortical layering in the Reeler mice is inverted, as is their hallmark. BrdU staining is all but absent in the cortex of wild-type mice, however, in contrast the Reeler cortex exhibits numerous positively stained cells. FIG. 5G and 5H show the alveus of wild-type and Reeler mice respectively. The number of BrdU positive cells is increased in the Reeler mice relative to wild-types. FIGS. 5I and 5K show low and high magnification of the anterior subventricular zone of the lateral ventricle wall in wild-type brain. The corresponding sections of Reeler brains (FIGS. 5J and L) exhibit a ventricle of reduced size, and an expanded subventricular zone packed with BrdU positive cells. The number of BrdU positive cells in Reeler mice is greatly increased compared to that of wild-type controls.

A recent study of adult cortical neurogenesis suggests that a proliferating progenitor may exist in the cortex that is stimulated to divide following focal cerebral ischemia (Zhang, Zhang et al. 2001). As shown in FIG. 5, BrdU positive cells were not observed in wild-type animals, however in Reeler cerebral cortices numerous positively stained cells were observed, suggesting that resident cortical stem cells may exist in Reeler cortices. Disruption of the hippocampal structure in Reeler mice is to such a degree that makes comparisons of numbers of hippocampal BrdU positive cells difficult. Comparison of the wild-type hippocampal field with that of the equivalent for the Reeler brain reveal a slight increase in the number of BrdU positive cells in the latter. The cells staining positively in the alveus of the Reeler mouse is intriguing, not only in their increased number relative to wild-type, but also in that they form a more pronounced elongated chain leading from the subventricular zone of the medial aspect of the posterior lateral ventricle to the hippocampal formation. The subventricular zone of the anterior lateral ventricle shows the most prominent increase in BrdU stained cells. To accommodate the large increase in cell number due to cell division, the subventricular zone is vastly expanded. Interestingly the lateral ventricle of the Reeler mouse is much reduced in size compared to wild-type mice, the reason for which is not known.

Overall, adult Reeler mice exhibit greater neural proliferation in the brain than wild-type mice, as exemplified by the hippocampus, cortex, alveus and anterior subventricular zone of the lateral ventricle.

In a further study the phenotypic fate of the BrdU population of cells was determined. The BrdU population of cells refers to the cells that have divided during the two weeks of treatment with BrdU. This study employed immunohistochemical techniques to detect BrdU incorporation and markers for doublecortin, a neuronal protein that regulates neuronal migration, and GFAP, a major intermediate filament protein expressed by astrocytes. Cells that double label for both BrdU and doublecortin or BrdU and GFAP represent cells that have divided and are maturing into neurons in the case of doublecortin or astrocytes in the case of GFAP. Analysis of neurogenic regions of the brain, such as the SVZ of the LVW and the hippocampus of Reeler mouse. brains reveal the presence of cells that positively double label for both BrdU/doublecortin and BrdU/GFAP. These results indicate that the proliferating population of cells in the SVZ of the LVW and hippocampus of the Reeler mouse can mature into both neurons and astrocytes.

### Example 3

### Expression of Gas6 and its Receptors Tyro3, Axl and Mer

Methods

A. Mouse Neurosphere Cultures

The anterior lateral wall of the lateral ventricle of 5-6 week old mice was enzymatically dissociated in 0.8 mg/ml hyaluronidase and 0.5 mg/ml trypsin in DMEM containing 4.5 mg/ml glucose and 80 units/ml DNase at 37° C. for 20 min. The cells were gently triturated and mixed with three volumes of Neurosphere medium (DMEM/F12, B27 supplement, 12.5 mM HEPES pH7.4) containing 20 ng/ml EGF (unless otherwise stated), 100 units/ml penicillin and 100 μg/ml streptomycin. After passing through a 70 μm strainer, the cells were pelleted at 160×g for 5 min. The supernatant was subsequently removed and the cells resuspended in Neurosphere medium supplemented as above, plated out in culture dishes and incubated at 37° C. Neurospheres were ready to be split approximately 7 days after plating.

To split neurosphere cultures, neurospheres were collected by centrifugation at 160×g for 5 min. The neurospheres were resuspended in 0.5 ml Trypsin/EDTA in HBSS (1×), incubated at 37° C. for 2 min and triturated gently to aid dissociation. Following a further 3 min incubation at 37° C. and trituration, 3 volumes of ice cold NSPH-media-EGF were added to stop further trypsin activity. The cells were pelleted at 220×g for 4 min, resuspended in fresh Neurosphere medium supplemented with 20 ng/ml EGF and 1 nM bFGF plated out and incubated at 37° C.

B. Human Neural Stem Cell (HNSC) Cultures

A biopsy from the anterior lateral wall of the lateral ventricle was taken from an adult human patient and enzymatically dissociated in PDD (Papain 2.5 U/ml; Dispase 1 U/ml; Dnase I 250 U/ml) in DMEM containing 4.5 mg/ml glucose and 37° C. for 20 min. The cells were gently triturated and mixed with three volumes of Human Stem Cell Plating Medium (HSCPM) (DMEM/F12; 10% FBS). The cells were pelleted at 250×g for 5 min. The supernatant was subsequently removed and the cells resuspended in HSCPM, plated out on fibronectin coated culture dishes and incubated at 37° C. in 5% CO₂. The following day the expansion of the culture was initiated by change of media to HSC culture media (DMEM/F12; BIT 9500; EGF 20 ng/ml; FGF2 20 ng/ml). The HSC were split using trypsin and EDTA under standard conditions. FBS was subsequently added to inhibit the reaction and the cells collected by centrifugation at 250×g for 5 min. The HSC were replated in HSC culture media.

C. RT-PCR

Mouse Neurosphere Cultures

The following primers pairs were designed to specifically identify the presence of Gas6, Tyro3, Axl and Mer gene expression in undifferentiated mouse neurospheres. Estimated band sizes are for each primer pair is indicated

Band size
(base pairs)

| | | |
|---|---|---|
| gas6 | CGTAGTCTAATCACGGGGGTCC | 272 |
| | (SEQ ID NO:33) | |
| | TCTTGCCTCTGCGATGAGGG | |
| | (SEQ ID NO:34) | |
| | AGCTGGAACGTAACCTTGTCA | 500 |
| | (SEQ ID NO:35) | |
| | GACCACCAACTGCTTCTTGAG | |
| | (SEQ ID NO:36) | |
| | AGCTGGAACGTAACCTTGTCA | 750 |
| | (SEQ ID NO:37) | |
| | GGTGCAGAAATCACCGATACT | |
| | (SEQ ID NO:38) | |
| axl | AACTTTCAGATGCAGGGGAGT | 400 |
| | (SEQ ID NO:39) | |
| | TAGCTCCGTAGGTTGTCTGGA | |
| | (SEQ ID NO:40) | |
| | AACTTTCAGATGCAGGGGAGT | 584 |
| | (SEQ ID NO:41) | |
| | GTGAGGAAGGAGCTTTTCCAG | |
| | (SEQ ID NO:42) | |
| tyro3 | TGGAACGGTCTGATGCTGGC | 350 |
| | (SEQ ID NO:43) | |
| | TGCTTGAAGGCGAACAATGG | |
| | (SEQ ID NO:44) | |
| | TGGAACGGTCTGATGCTGGC | 550 |
| | (SEQ ID NO:45) | |
| | CCAAGTTCCGAAGCAGGCAG | |
| | (SEQ ID NO:46) | |
| mer | CATAGCCAGTGTGCAGCGCTC | 354 |
| | (SEQ ID NO:47) | |
| | GATATGTACACCCTTGGACACCG | |
| | (SEQ ID NO:48) | |
| | CATAGCCAGTGTGCAGCGCTC | 581 |
| | (SEQ ID NO:49) | |
| | AATTAGCCAGGGCTTGCAGC | |
| | (SEQ ID NO:50) | |

Mouse neurospheres were prepared from the LVW as stated above. 3 days after the first split, the neurospheres were harvested and total RNA isolated using Qiagen's RNeasy Mini Kit according to the manufacturer's instructions. Life Technologies One-Step RT-PCR Kit was used to detect the presence of gas6, tyro3, axl and mer mRNA. Briefly, 12.5 ng of total RNA was used in each reaction, with an annealing temperature of 55° C. or 60° C. To ensure genomic contamination of the total RNA did not give rise to false positive results, an identical reaction in which the RT-taq polymerase mix was replace by taq polymerase alone and was run in parallel with the experimental RT-PCR. The reactions were electrophoresed on a 1.5% agarose gel containing ethidium bromide and the bands visualised under UV light. Bands corresponding to the estimated length of PCR products of the desired genes were cloned into the cloning vector pGEM-Teasy and sequenced to verify their identity.

Human Neural Stem Cell Cultures

The following primer pairs were designed to specifically identify the presence of gas6, axl, tyro3 gene expression in HNSC cultures. Estimated band sizes for each primer pair are given below:

Band size
(base pairs)

| | | |
|---|---|---|
| gas6 | ACACCAAAAACTCAGGCTTCGCC | 378 |
| | (SEQ ID NO:51) | |
| | ACGCAAAGCCCTCGTCACAGAG | |
| | (SEQ ID NO:52) | |
| | ACACCAAAAACTCAGGCTTCGCC | 427 |
| | (SEQ ID NO:53) | |
| | CCCTGCAGACACTCGTCCACATCT | |
| | (SEQ ID NO:54) | |
| axl | GCCAGGAACTGCATGCTGAAT | 331 |
| | (SEQ ID NO:56) | |
| | ACCGCGACATCAAGGCATA | |
| | (SEQ ID NO:57) | |
| tyro3 | CCGGTGAAGCTGACAGTGTCTCA | 425 |
| | (SEQ ID NO:58) | |
| | AAACAGATGGAGAGGGAGCGG | |
| | (SEQ ID NO:59) | |
| | CCGGTGAAGCTGACAGTGTCTCA | 474 |
| | (SEQ ID NO:60) | |
| | GTGAGCTTCACAGGAAAACATGGTG | |
| | (SEQ ID NO:61) | |

HNSC were prepared and cultured as stated above. Total RNA isolated using Qiagen's RNeasy Mini Kit according to the manufacturer's instructions and DNase treated using Ambion DNase I and according to protocol. Life Technology's One-Step RT-PCR Kit was used to detect the presence of gas6, axl and tyro3 mRNA. Briefly, 50 ng of total RNA was used in each reaction, with an annealing temperature of 55° C. To further ensure that genomic contamination of the total RNA did not give rise to false positive results, an identical reaction in which the RT-Taq polymerase mix was replaced by Taq polymerase alone and was run in parallel with the experimental RT-PCR. The reactions were electrophoresed on a 1.5% agarose gel containing ethidium bromide and the bands visualized under UV light. Bands corresponding to the estimated length of PCR products of the desired genes were cloned into the cloning vector pGEM-Teasy and sequenced to verify their identity.

D. Flow Cytometry

Cultured neurospheres from early passages were trypsinized and resuspended into a single cell suspension as described previously. The cells were then spun down at 200×g for 5 min and resuspended in primary antibody (1 μg of antibody in 100 μl of PBS with 3% FBS and 0.09% sodium azide “Stain Buffer”) and incubated for 20 minutes at room temperature. The respective primary antibodies used were goat anti-mouse Tyro3, goat anti-mouse Axl and goat anti-mouse Mer (R&D Systems). After incubation, the cells were washed in 1 ml of Stain Buffer followed with centrifugation at 200×g for 5 min. The cells were then resuspended in 1 μg of secondary antibody (FITC labeled rabbit anti-goat IgG) in 100 μl Stain Buffer and incubated for 20 min at room temperature in dark. Following incubation, the neurospheres were washed as described previously and resuspended in 500 μl of Stain Buffer. 5 μl of 7-AAD was added to allow for dead cell exclusion. Analysis was carried out on a FACSCalibur (Becton Dickinson) using a 488 nm blue laser and CellQuest Pro software. Parameters measured include FSC, SSC and FITC fluorescence intensity measured by a 530/30 BP filter, as well as 7-AAD fluorescence intensity measured by a 670 LP filter. 10,000 events from each sample were collected as defined by regions set in the dot plot.

E. Radioactive In Situ Hybridization

In situ hybridization was carried out essentially using published methods (Zachrisson et al., 1999). In brief, sections (14 μm) of mice brains were cut on a cryostat at −17° C., thawed onto microscope slides (Superfrost Plus, BDH, UK) and fixed in 4% formaldehyde for 5 min, deproteinated and dehydrated. To detect Tyro-3, Axl and Gas6 mRNA, murine antisense cRNA probes were transcribed from plasmids encompassing bp 271-620 of the Tyro-3 cDNA, bp 305-890 of the Axl cDNA and bp 1147-1646 of the Gas6 cDNA, and concurrently [α-³⁵S]-UTP-labeled. After hybridization, the sections were treated with RNaseA and washed in 4×saline sodium citrate, followed by a high stringency wash at 70° C. for 30 min in 0.1×SSC. All wash steps included the addition of 1 mM DTT. The sections were dehydrated in an ascending series of ethanol concentrations, dried overnight and dipped in Kodak NTB-2 nuclear rack emulsion, diluted 1:1, exposed for six weeks, developed, fixed and counterstained with hematoxylin The specificity of the hybridization was tested using a sense probe transcribed from the same plasmid in a set of hybridizations. No hybridization signal was obtained under this condition (data not shown). The emulsion-dipped sections were analysed manually, using a microscope. Due to the similar size of the relevant cell types, cellular profiles with more than 10 exposed silver grains were considered positive. The results are given as a percentage hybridization positive cellular profiles.

The regions quantified were:

the anterior ependymal cell layer of striatum (EpStr), anteriorposterior bregma 1.1 mm, lateral 0.72 mm, dorsalventral bregma 2.75-4 mm.

the anterior subependymal layer of striatum (SubEpStr), anteriorposterior bregma 1.1 mm, lateral 0.72 mm, dorsalventral bregma 2.75-4 mm.

the anterior ependymal cell layer of septum (EpSpt), anteriorposterior bregma 1.1 mm, lateral 0.48 mm, dorsalventral bregma 2.75-4 mm.

the anterior subependymal layer of septum (SubEpSpt), anteriorposterior bregma 1.1 mm, lateral 0.48 mm, dorsalventral bregma 2.75-4 mm.

the anterior subventricular zone (SVZa), anteriorposterior bregma 1.1 mm, lateral 0.8 mm, dorsalventral bregma 2.5 mm

the posterior ependymal cell layer of striatum (EpStr(p)), anteriorposterior bregma −1.7 mm, lateral 2.7 mm, dorsalventral bregma 2.0-2.75 mm

the posterior subependymal layer of striatum (SubEpStr(p)), anteriorposterior bregma −1.7 mm, lateral 2.7 mm, dorsalventral bregma 2.0-2.75 mm

the granular cell layer in the dentate gyrus of hippocampus (DenGyr), anteriorposterior bregma −1.7 mm, lateral 0.25-1.25 mm, dorsalventral bregma 2 mm

Expression of Gas6, Tyro3, Axl and Mer mRNA in Cultured Non-adherent Mouse Neurospheres

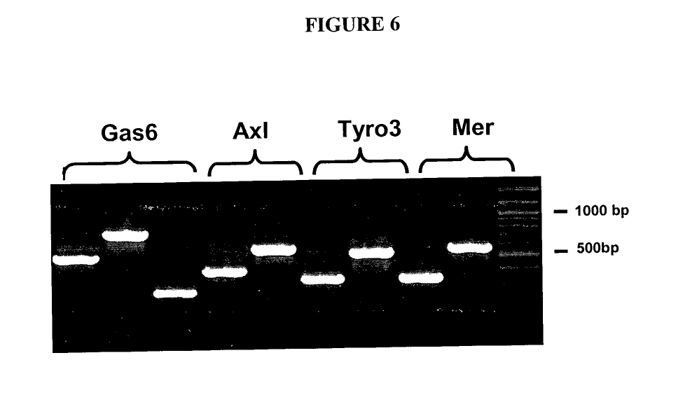

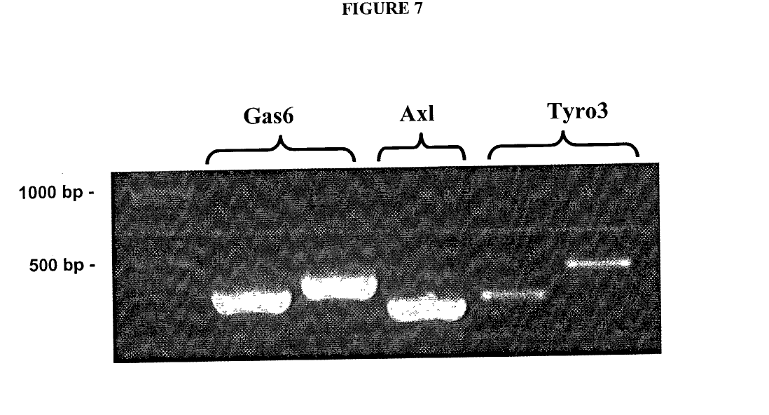

RT-PCR was performed on total RNA prepared from undifferentiated mouse neurospheres using primer pairs for the above specified genes as described the methods. The bands indicated correspond to the bands of the desired size (gas6 [lane 1, 500 bp; lane 2, 750 bp; lane 3, 272 bp], axl [lane 4, 400 bp; lane 5, 585 bp], tyro3 [lane 6, 350 bp; lane 7, 550 bp; lane 7], mer [lane 8, 354 bp; lane 9, 581 bp]) (FIG. 7). Sequencing of these bands confirmed that they represent correct product. The resulting PCR products were subcloned and sequenced, verifying that they represent correct product (FIG. 6). A parallel control experiment using no reverse transcriptase, only Taq polymerase ruled out false positive bands through genomic contamination.

Expression of Gas6, Axl and Tyro3 mRNA in Cultured Adult Human Neural Stem Cells

RT-PCR was performed on total RNA prepared from cultured adult HNSC using primer pairs specific for the above genes. The bands indicated correspond to the bands of the desired size (gas6 [lane 1378 bp; lane2 331 bp], axl [lane3 421 bp], tyro3 [lane4 425 bp; lane5 474 bp]) (FIG. 7). Sequencing of these bands confirmed that they represent correct product. A parallel control experiment without using any reverse transcriptase, only taq polymerase, ruled out false positive,bands through genomic contamination.

Tyro3, Axl and Mer Protein Levels in Cultured Non-adherent Mouse Neurospheres

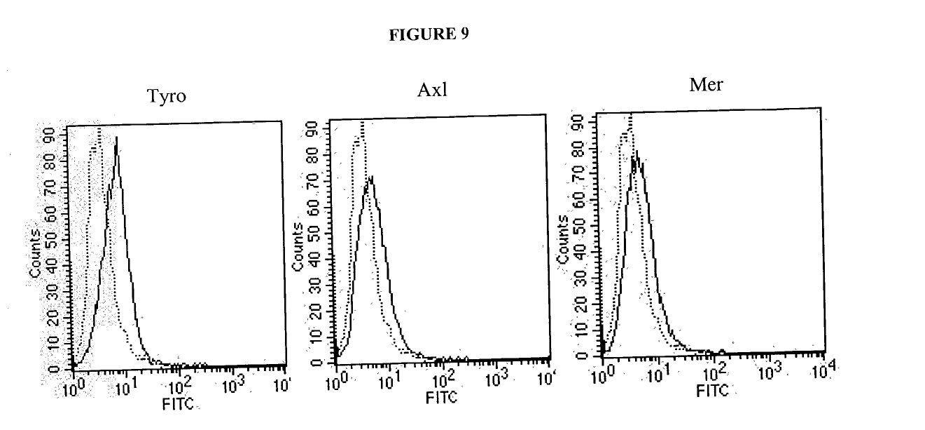

Flow cytometry was performed on cultured mouse neurospheres using specific antibodies directed toward the N-terminal of each independent receptor (R&D Systems) as described above. Dotted line represents control staining and unbroken line represents receptor specific staining (FIG. 9).

Expression of Gas6, Tyro3 and Axl mRNA in Neurogenic Regions of the Adult Mouse Brain

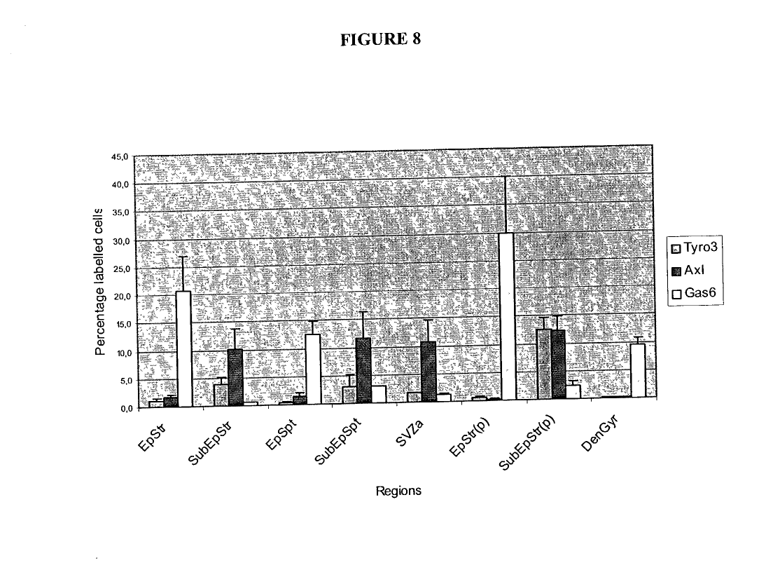

FIG. 8 represents the mRNA expression of Gas6 and its receptors. Gas6 mRNA signal was found predominantly in the striatal ependymal cell layer lining the lateral ventricle with 29.9% mRNA positive cells in the posterior part (EpStr(p)) and 20.7% mRNA positive cells in the anterior part (EpStr). In addition, 12.5% of the cells in the septal ependymal cell layer lining the lateral ventricle (EpSpt) were positive for Gas6 mRNA. The granular cell layer of the dentate gyrus of the hippocampus (DenGyr) expressed Gas6 mRNA in 9.4% of the cells. Only low levels were found in the remaining examined regions with 0.7% Gas6 mRNA positive cells of the subependymal cells of the anterior striatum (SubEpStr), 2.3% of the subependymal cells of the posterior striatum (SubEpStr(p)), 3.0% positive cells of the subependymal cells in the septum (SubEpSpt) and 1.3% in the anterior subventricular zone (SVZa).

In contrast, Axl mRNA expressing cells were expressed in very few cells of the ependymal cell layer with 1.7% Axl mRNA positive cells in EpStr, 0.3% positive cells in EpStr(p) and 1.5% positive cells in EpSpt. Moreover, only 0.2% of cells in DenGyr expressed Axl mRNA. On the other hand, all subependymal structures examined expressed moderate Axl mRNA levels with 10.0% of the cells positive in SubEpStr, 12.2% positive cells in SubEpStr(p), 11.6% positive cells in SubEpSpt and 10.8% Axl mRNA positive cells in SVZa.

Similar to Axl mRNA, Tyro-3 mRNA expressing cells were rarely seen in the ependymal cell layer with 1.1% Tyro-3 mRNA positive cells in EpStr, 0.6% positive cells in EpStr(p) and 0.4% positive cells in EpSpt. Moreover, only 0.2% of cells in DenGyr expressed Tyro-3 mRNA. Of the subependymal structures Tyro-3 mRNA was expressed in 3.9% of the cells in SubEpStr, 12.5% positive cells in SubEpStr(p), 3.0% positive cells in SubEpSpt and 1.8% Tyro-3 mRNA positive cells in SVZa.

### Example 4

### In Vitro Experiments to Define the Therapeutic Potential of Gas6 and its Receptors Axl/Tyro3/Mer

Methods

A. Mouse Neurosphere Cultures

The anterior lateral wall of the lateral ventricle of 5-6 week old mice was. enzymatically dissociated in 0.8 mg/ml hyaluronidase and 0.5 mg/ml trypsin in DMEM containing 4.5 mg/ml glucose and 80 units/ml DNase at 37° C. for 20 min. The cells were gently triturated and mixed with three volumes of Neurosphere medium (DMEM/F12, B27 supplement, 12.5 mM HEPES pH7.4) containing 20 ng/ml EGF (unless otherwise stated), 100 units/ml penicillin and 100 μg/ml streptomycin. After passing through a 70 μm strainer, the cells were pelleted at 160×g for 5 min. The supernatant was subsequently removed and the cells resuspended in Neurosphere medium supplemented as above, plated out in culture dishes and incubated at 37° C. Neurospheres were ready to be split approximately 7 days after plating.

To split neurosphere cultures, neurospheres were collected by centrifugation at 160×g for 5 min. The neurospheres were resuspended in 0.5 ml Trypsin/EDTA in HBSS (1×), incubated at 37° C. for 2 min and triturated gently to aid dissociation. Following a further 3 min incubation at 37° C. and trituration, 3 volumes of ice cold NSPH-media-EGF were added to stop further trypsin activity. The cells were pelleted at 220×g for 4 min, resuspended in fresh Neurosphere medium supplemented with 20 ng/ml EGF and 1 nM bFGF plated out and incubated at 37° C.

B. Intracellular ATP Assay

Intracellular ATP levels have previously been shown to correlate to cell number (Crouch et al., J. Immunol. Methods, 160 (1): 81-88, 1993). Mouse neurospheres, cultured as described above, from passage 2, were seeded in DMEM/F12 supplemented with B27 into a 96-well plate as single cells (10000 cells/well) to which rhGas6 was added at the concentrations indicated. After 6 days incubation, intracellular ATP was measured using the ViaLight kit (BioWhittaker) according to the manufacturer's instructions. For analysis of intracellular ATP levels in adherent cultures, adult mouse neural stem cells isolated and expanded as described above, were seeded as 30,000 cells/well into a 96 well poly-D-lysine coated plate in neurosphere medium supplemented with B27 and 1% FBS, and allowed to attatch overnight. Next day, rhGAS6 was added at the concentrations indicated and cells were incubated for 4 days. Intracellular ATP was measured as above using the ViaLight kit (BioWhittaker) according to the manufacturer's instructions.

C. Production of Stable HEK293 Cells Expressing Recombinant HumanGas (rhGas6)

Human Gas6 cDNA was transfected into human embryonic kidney (HEK293) cells using LipoFectAMINE2000 (Invitrogen) according to manufacturers instructions. Colonies with stable integration events were selected with G418 (500 μg/ml) and isolated using the cloning-ring method. Isolated clones were assayed for hGas6 expression with western blotting of the conditioned media using an anti-hGas6 antibody (R&D Systems). Clones showing highest expression were expanded for production. For production, cells were propagated adherent in DMEM/10% FCS with the appropriate selection antibiotics. When the cells reached near-confluency, the plates were washed twice with PBS and the media was changed to DMEM:F12 mix supplemented with penicillin-steptomycin and 5 μg/ml vitamin K (Konakion Novum, Roche). After 3-4 days the medium was collected and replaced for another 2-3 days. The collected media were clarified by centrifugation.

D. Purification of rhGas6

Recombinant hGas6 was purified using pseudoaffinity chromatography followed by size-exclusion chromatography. Before purification, 50 mM Tris-Cl pH 7.5, 10 mM EDTA and 0.01% Tween 80 was added to the conditioned medium. The medium was then applied to a HiTrap Q HP-column at 8 ml/min at RT. The column was washed with 20 mM Tris-Cl pH7.5/230 mM NaCl/1 mM EDTA at 5 ml/min at RT. The Gas6 was eluted using 20 mM Tris-Cl pH7.5/200 mM NaCl/10 mM CaCl₂ at 2 ml/min at RT. The fractions containing rGas6 were concentrated using ultrafiltration (30 k Omega, Pall). The concentrated rGas6 was injected (max 500 μl/run) into Superdex200 HR10/30 column equilibrated in 10 mM HEPES, pH 7.4/140 mM NaCl/3 mM KCl/1.4 mM CaC₂/0.8 mM MgCl₂/0.005% Tween 80 and run at 0.3 ml/min at RT. The rGas6-peak, eluted at 13.8 ml, was collected and stored frozen in aliquots at −70° C. The identity of the purified protein was confirmed by N-terminal amino-acid sequencing and western blotting with anti-Gas6 antibodies. The sequence AFQVF—A was consistent with the observed post-translational cleavage reported in the literature. The standard procedure used yields a blank amino-acid on encountering a γ-carboxylated glutamic acid residue. Thus, the lack of two observable residues between the phenylalanine and alanine residues is consistent with γ-carboxylation of the glutamic acid residues located here in the expected sequence. The protein was quantitated spectrophotometrically using absorbance at 280 nm and by ninhydrin amino-acid analysis. In spectrophotometric quantitation extinction coefficients were calculated from the sequence (for human Gas6 ε_{mg/ml}=0.912). The two methods were found to give similar results. The purity was estimated from silver-stained gradient SDS-PAGE gel to be at or above 99%.

E. Tyrosine Phosphorylation Assay (DELFIA)

The adult mouse neurospheres were dissociated with trypsin (Gibco) into single cells and transferred to FBS (Gibco) coated 96 well tissue culture plates (Costar) in Neurosphere medium without growth factors, supplemented with 1% Fetal Calf Serum (Gibco). The cells were incubated over night to attach. The next day, the medium was replaced with fresh Neurosphere medium without growth factors. The cells were incubated for an additional 3 days. Cells were then washed in PBS (Gibco) and incubated in DMEM:F12 without supplements for 6 h. The cells were treated with 3 nM Gas 6 (NeuroNova) or plain DMEM:F12 for 5 min and fixated with 4% Formaldehyde (Sigma) in PBS, permiabilised with 0,1% Triton X-100 (Sigma) in PBS and labeled with 0, 5 ug/ml rabbit anti P-Tyr PT66 Eu (Perkin Elmer). Enhancement Soulution (Perkin Elmer) was added, incubated 90 min 300 rpm, the europium fluorescence (excitation at 340 nm and emission at 615 nm) was measured over a 400 us time period after an initial delay of 400 us with a Victor² spectrofluorimeter.

F. Differentiation

Adult mouse neurospheres were dissociated with trypsin (Gibco) into single cells and transferred to poly-D-Lysine plates (BD) in neurosphere medium without growth factors, supplemented with 1% Fetal Calf Serum (Gibco). The cells were incubated overnight in order to adhere to the plate. The next day, the medium was replaced with fresh medium, without growth factors, supplemented with 3 nM Gas6 and incubated for a total of 3 days.

G. Immunocytochemistry

The cells were washed twice with PBS (Gibco), fixed with 4% Formaldehyde (Sigma) for 15 min and permeabilised in 0,1% Triton X-100 (Sigma) in PBS for 20 min at RT. The cells were then labeled with mouse monoclonal anti β-III Tubulin (1:500 Promega), a neuronal marker antibody, and rabbit anti GFAP (1:500 Sigma), a glial marker antibody against glial fibrillary acidic protein (GFAP). Primary antibodies were visualized with anti mouse Texas-Red and anti rabbit FITC (1:100 Vector Laboratories). All antibodies were diluted in PBS with 0,1% Triton X-100. Nuclei were stained with 300 nM DAPI (Molecular Probes) Smin.

Effect of Gas6 on Proliferation of Cultured Non-adherent Mouse Neurospheres

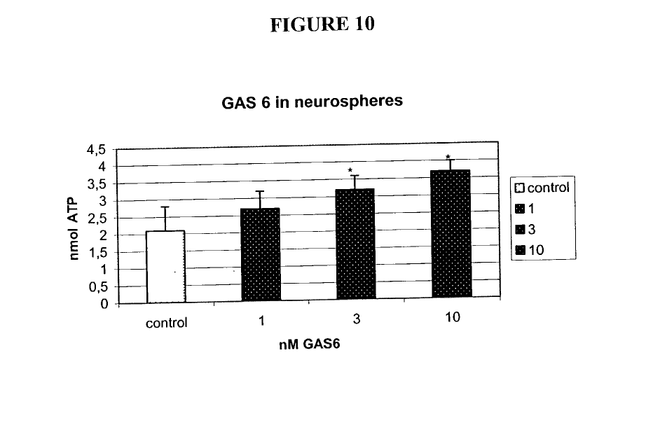

To determine the effect of Gas6 on neural stem cells in culture, adult mouse neural stem cells isolated and expanded as described above, were cultured in neurosphere medium supplemented with varying concentrations of Gas6 under non-adherent conditions. Briefly, cells were seeded as 10,000 cells/well in 96 well plates in medium supplemented with B27 and rhGAS6 was added at the concentrations indicated (1-10 nM). Cells were incubated for 6 days and ATP was measured according to the method of the manufacturer. ATP correlates with cell number. Mean +/−s.d. n=4, rhGAS6 vs. control p<0,05. As demonstrated in FIG. 10, a statistically significant, dose dependent increase in intracellular ATP levels was observed in Gas6 versus mock treated cells.

Effect of Gas6 on Proliferation of Adherent Neural Stem Cell/progenitor Cultures

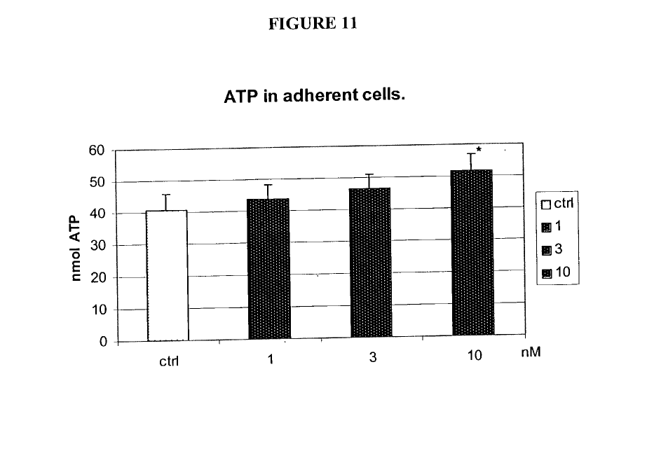

To determine the effect of Gas6 on neural stem cells grown as a more differentiated adherent culture, adult mouse neural stem cells isolated and expanded as described above, were seeded as 30,000 cells/well into a 96 well poly-D-lysine coated plate in neurosphere medium supplemented with B27 and 1% FBS, and allowed to attatch overnight. On the next day, rhGAS6 was added at the concentrations indicated (1-10 nM) and cells were incubated for 4 days. ATP was measured according to the method of the manufacturer. ATP correlates with cell number. Mean +/−s.d. n=4, rhGAS6 vs. control p<0, 05 (FIG. 11). As demonstrated in FIG. 11, Gas6 had a concentration dependent proliferative effect on cells grown as a more differentiated adherent culture (possibly progenitor cells) on poly-D-lysine.

Effect of Gas6 on Induction of Tyrosine Phosphorylation in Adherent Mouse Neural Stem Cells/progenitor Cultures

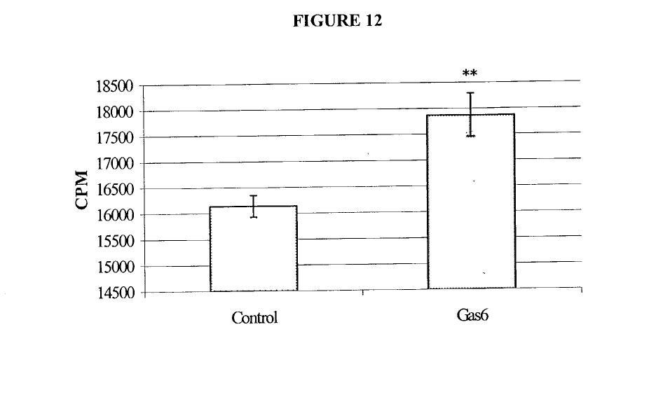

As measured quantitatively (FIG. 12), compared to mock treated cells, treatment with Gas6 resulted in statistically significant increase in tyrosine phosphorylation in adult mouse neural stem cell cultures grown as a more differentiated adherent culture (possibly progenitor cells) on poly-D-lysine (** represents p<0.001). The increase in tyrosine phosphorylation in response to Gas6 demonstrates that Gas6 mediated signal transduction pathways are present and functional in cultured mouse neurospheres further supporting a role for Gas6 and its receptors in the regulation of neural stem cell/neural progenitor function.

Exposure to Gas6 Results in A Proportional Increase in β-III Tubulin Positive Cells in Gas6 Treated Versus Control Treated Cells

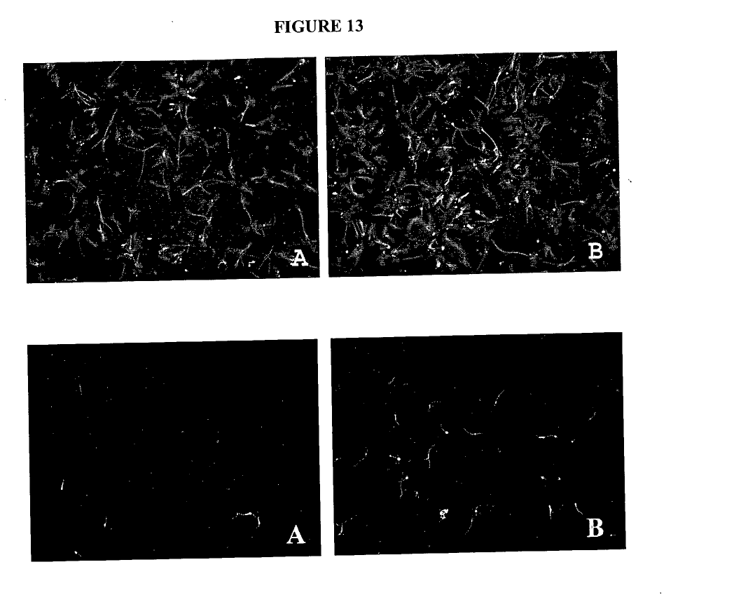

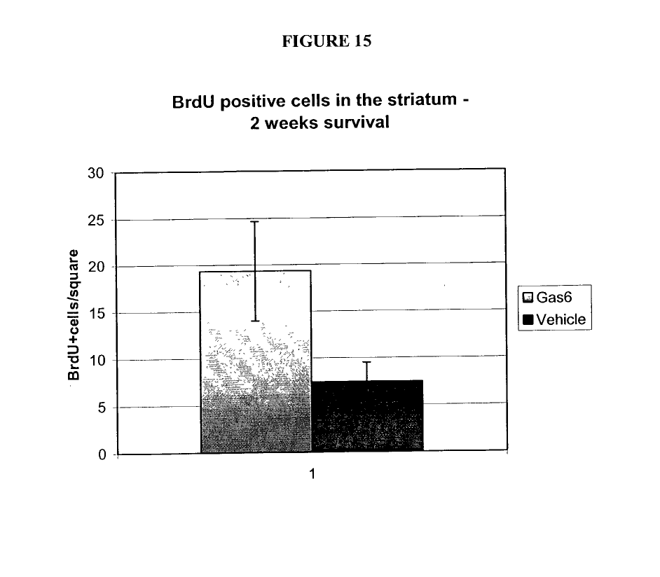

The upper panels (A and B) of FIG. 13 represent cell cultures double immunostained with anti B-III Tubulin antibody and anti-GFAP antibody. The lower panels (A and B) of FIG. 13 represent cell cultures immunostained with anti B-III Tubulin antibody. As measured qualitatively, compared to the control (FIG. 13, upper panel A), treatment with Gas6 (FIG. 13, upper panel B) significantly increases the total number of cells in culture resulting in an increased number of cells staining positively with the neuronal specific, anti B-III Tubulin antibody (FIG. 15, compare lower panel A (control) versus B (Gas6)).

Taken together, this data shows that Gas6 triggers proliferation resulting in a corresponding increase in the generation of new neurons. Therefore, Gas6 is a neurogenic factor in vitro.

### Example 5

### In Vivo Analysis of Gas6 on Neural Proliferation

Methods

A. In Vivo Studies

Animals were kept under standard housing conditions at least 7 days after delivery (12 hrs light/dark cycle; temp.: 22° C; humidity: 45-60%). Operations were performed with male rats: Wistar Unilever, weight before pump implantation: 317.7±4.15 g (2-weeks survivors) and 304.5±2.11 g (4-weeks survivors); weight before decapitation: 350±4.6 g (2-weeks survivors). Anaesthesia was induced with a mixture of 4% halothane in oxygen/nitrous oxide (1:1). The animals were fixed in a stereotaxic instrument (Stoelting) and halothane reduced to 3% during implantation of pump and 2.0-2.5% during removal of pump. For implantation the skin above the skull was removed to expose bregnia and midline. After removal of the periosteum a stainless steel screw was inerserted without piercing the dura. A hole was drilled over the lateral ventricle for insertion of the infusion cannula (0.08 cm posterior to bregma; 0.17 cm lateral to midline; and 0.45 cm below dura). An incision was made in the skin on the neck and the pump (which was connected to the brain infusion cannula) was placed in the midscapular region (Alzet osmotic mini-pumps; model 2002; 0.5 μl/h; 14 days; Lot.: 10025-01 with brain infusion kit II; Lot.: 10025-01; filled with: BrdU-solution: 1 mg BrdU/ml (Lot.: 71K1172; Sigma) aCSF (148 mM NaCl, 3 mM KCl, 1.4 mM CaCl2, 0.8 mM MgCl₂, 1.5 mM Na₂HPO₄, 0.2 mM NaH₂PO₄, pH 7.4, sterilfiltration; stored at −20° C.; before usage: 100 μg/ml rat serum albumin (Sigma), 50 μg/ml Gentamycin (Sigma); BrdU added at 37° C. and ultra-sonication, 66.7 μg/ml rhGas6; pump filled with 200 μl of aforementioned solution, connected via tubing to flow moderator (also filled with aforementioned solution); put in NaCl (0.9%) solution in water bath (37° C.) for 2-5 hrs (note time point).

For removal of brain, animals were anesthetized by intraperitoneal injection of 6 ml chloralhydrate solution (4 g/100 ml). In deep narcosis the rats were transcardially perfused with saline for 3-5 min and afterwards with ca. 60 ml paraformaldehyde-solution (4%). The brains were stored over night in this solution, then transferred into sucrose solution (30%) and stored in refrigerator until they are on the bottom. The olfactory bulbs were separated and sliced. The rest of the brain was frozen at −80° C. and contralateral side was marked with a notch.

The brains were cut on a cryotom. The ipsilateral olfactory bulb was cut sagitally and the rest of brain was cut coronally in 40 μm sections. The sections were stored in CPS-solution (Ethylenglycol/Glycerol/PBS 1:1:2) at −20° C. during project and afterwards at 4-6° C.

For immunohistochemistry (detection of BrdU+-cells with diaminobenzidine (DAB)) slices were washed and endogenous peroxidase activity blocked with a solution containing methanol, PBS and H₂O₂. Interrupted by washing steps, denaturation was performed with HCL-solution (2N), and unspecific binding blocked with donkey normal serum for 1 hr. The slice was incubated with the primary antibody (monoclonal rat anti BrdU IgG2a (Lot.: PC 01H250-2 (Harlan) 1:500) over night. Afterwards the secondary antibody (donkey anti rat IgG-biotinylated (Lot.: 52149 (Dianova); 1:5000) was applied and incubated for 1 hr and then the slice incubated with solutions from APB-Kit (1:400; Vectastain).

For double labeling studies with BrdU and the neuronal marker NeuN, washed slices were incubated in HCL solution (2N) for 1 hr. With washing-steps in between, blocking was performed with goat-serum and slices incubated with primary antibodies (mouse anti NeuN (1:250; Lot.: 2204037; Chemicon) and rat anti-BrdU IgG 2a (1:100; Lot.: PC 02a2503; Harlan) over night. Afterwards slices were incubated for 2 hrs with secondary antibodies: 1. goat anti rat IgG2a-FITC (1:200; Lot.: GG2A-25F-1; Dunn, Bethyl) and goat anti mouse IgG1 Alexa Fluor 568 (1:200; Lot.: 73B1-1; MoBiTec). For controls, slices were treated with secondary antibody only and did not show any obvious labeling.

Quantification was performed on a Zeiss LSM Pascal (double labeling) and on a Leica Microscop using Zeiss-Axiovision Software for quantification. Multitracking was used to avoid bleeding. For statistics Mann Whitney test was applied (using GraphPad Prism Software).

In Vivo Proliferation Analysis

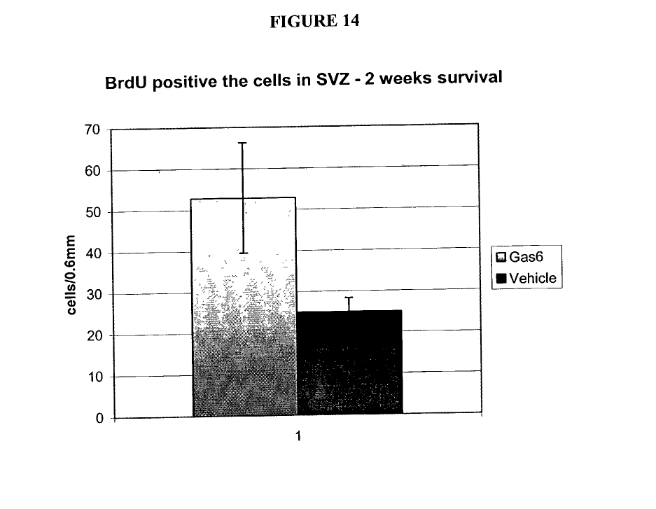

Male rats (12 hours light/dark regime; feeding and drinking ad libitum; 5 animals in standard cage) were infused (Alzet minipumps) in the left lateral ventricle with human recombinant Gas6 for 14 days at a daily dose of 0.8 μg/day (6 animals in treated, 8 animals in control group). Bromodeoxyuridine (BrdU) was also included in the infusion vehicle (artifical cerebrospinal fluid) to enable measurement of proliferation by quantitation of BrdU incorporation in the DNA. Animals were sacrificed at 14 days after start of treatment and brains were dissected and prepared for sectioning and immunohistochemistry as described above (see also Pencea V et al., J. Neurosci Sep. 1 (2001), 21(17):6706-17). Proliferation was measured by BrdU incorporation and diaminobenzidine (DAB) staining of HRP conjugated secondary antibodies. Cells were counted in a phase contrast microscope (FIG. 14 and 15). Neural phenotype was assessed by staining for the neuronal marker NeuN by immunocytochemistry using a fluorescence readout as described above (see also see Pencea V et al., J. Neurosci Sep. 1 (2001), 21(17):6706-17). In double staining analyses designed to measure neurogenesis, BrdU and NeuN were quantitated by fluorescence (above) and counting of cells was done in a confocal microscope. A number of brain regions were analysed (subventricular zone and striatum).

FIG. 14 represents the number of BrdU positive cells in the subventricular zone after two weeks infusion with rhGas6. Quantitation of BrdU positive cells in the subventricular zone indicates an increase in the number of proliferating cells in Gas6 treated versus control animals. The data represents the mean +/−s.d. *, p<0.05 compared to vehicle.

FIG. 15 represents the number of BrdU-labeled cells in the striatum after two weeks infusion with rhGas. Quantitation of BrdU positive cells in the striatums indicates an increase in the number of proliferating cells in Gas6 treated versus control animals. The data represents the mean +/−s.d. *, p<0.05 compared to vehicle. Double staining analysis revealed that the ratio of NeuN positive cells to BrdU positive cells was similar in that there was no significant difference.

In summary, ICV infusion of Gas6 in healthy rats results in a significant increase in the number of proliferating cells in the regions analysed (subventricular zone and striatum). Also, ICV infusion of Gas6 in healthy rats does not significantly change the ratio of NeuN/BrdU. Taken together, it is shown that Gas6 is plays an integral role as a neurogenic factor in vivo.

### Example 6

### Biopolymer Sequences

The DNA and protein sequences referenced in this patent are as listed below.

| | |
|---|---|
| GenBank Accession Number | Description |

A. Reelin

| | |
|---|---|
| NM_005045 | Homo sapiens reelin (RELN), mRNA |
| NM_011261 | Mus musculus reelin (Reln), mRNA |

Splice Variants

Lambert de Rouvroit C, Bernier B, Royaux I, de Bergeyck V, Goffinet A M. Related Articles,
Links Evolutionarily conserved, alternative splicing of reelin during brain development. Exp
Neurol. April 1999;156(2):229-38.

B. VLDLR

| | |
|---|---|
| NM_003383 | Homo sapiens very low density lipoprotein receptor |
| | (VLDLR), mRNA |
| NM_013703 | Mus musculus very low density lipoprotein receptor |
| | (Vldlr), mRNA |

C. ApoER2

| | |
|---|---|
| NM_004631 | Homo sapiens low density lipoprotein receptor-related |
| | protein 8, apolipoprotein e receptor (LRP8), transcript |
| | variant 1, mRNA |
| NM_033300 | Homo sapiens low density lipoprotein receptor-related |
| | protein 8, apolipoprotein e receptor (LRP8), transcript |
| | variant 2, mRNA |
| NM_017522 | Homo sapiens low density lipoprotein receptor-related |
| | protein 8, apolipoprotein e receptor (LRP8), transcript |
| | variant 3, mRNA |
| NM_053073 | Mus musculus low density lipoprotein receptor-related |
| | protein 8, apolipoprotein e receptor (Lrp8), mRNA |

D. Dab1

| | |
|---|---|
| NM_021080 | Homo sapiens disabled homolog 1 (Drosophila) (DAB1), mRNA |
| Y08379 | M. musculus mRNA for mDab555 protein |
| Y08380 | BB466494 RIKEN full-length enriched, 12 days embryo eyeball |
| | Mus musculus cDNA clone D230009F07 3′ similar to Y08380 |
| | M. musculus mRNA for mDab271 protein, mRNA sequence |
| Y08381 | BB540098 RIKEN full-length enriched, 0 day neonate eyeball |
| | Mus musculus cDNA clone E130103G05 3′ similar to Y08381 |
| | M. musculus mRNA for mDab217 protein, mRNA sequence |
| Y08382 | M. musculus mRNA for mDab protein, exon, splice variant |

E. Gas6 (Growth arrest specific gene 6)
Mouse Gas6 mRNA SEQUENCES (3)

| | |
|---|---|
| NM_019521 | Mus musculus growth arrest specific 6 (Gas6), mRNA |
| BC005444 | Mus musculus, growth arrest specific 6, clone MGC:6124 |
| | IMAGE:3592398, mRNA, complete cds |
| X59846 | M. musculus GAS 6 mRNA associated with growth-arrest |

Human Gas6 mRNA SEQUENCES (3)

| | |
|---|---|
| NM_000820 | Homo sapiens growth arrest-specific 6 (GAS6), mRNA |
| AK092028 | Homo sapiens cDNA FLJ34709 fis, clone MESAN2003101, highly |
| | similar to Homo sapiens growth-arrest-specific protein (gas) |
| | mRNA |
| L13720 | Homo sapiens growth-arrest-specific protein (gas) mRNA, |
| | complete eds |

F. Tyro3: TYRO3 protein tyrosine kinase 3
(Aliases; Brt, Dtk, Rse, Sky, Tif, Etk-2)
Mouse Tyro3 mRNA SEQUENCES (8)

| | |
|---|---|
| D17393 | Mouse mRNA for protein tyrosine kinase, complete cds |
| U18343 | Mus musculus putative growth factor receptor tyrosine kinase |
| | isoform B (tyro3) mRNA, complete cds |
| U18342 | Mus musculus putative growth factor receptor tyrosine kinase |
| | isoform A precursor (tyro3) mRNA, complete cds |
| U05683 | Mus musculus receptor-type tyrosine kinase (rse) mRNA, |
| | complete cds |
| U18933 | Mus musculus receptor tyrosine kinase (Dtk) mRNA, complete |
| | cds |
| X78103 | M. musculus Tyro 3 mRNA for tyrosine kinase |
| NM_019392 | Mus musculus TYRO3 protein tyrosine kinase 3 (Tyro3), mRNA |
| AB000828 | Mouse mRNA for receptor tyrosine kinase, complete cds |

Human Tyro3 mRNA SEQUENCES (5)

| | |
|---|---|
| D17517 | Human sky mRNA for Sky, complete cds |
| NM_006293 | Homo sapiens TYRO3 protein tyrosine kinase (TYRO3), |
| | mRNA |
| U18934 | Human receptor tyrosine kinase (DTK) mRNA, complete |
| | cds |
| U05682 | Human receptor-type tyrosine kinase (rse) mRNA, |
| | complete cds |
| D50479 | Homo sapiens mRNA for protein-tyrosine kinase, |
| | complete cds |

Rat Tyro3 mRNA SEQUENCES (2)

| | |
|---|---|
| NM_017092 | Rattus norvegicus Bruton agammaglobulinemia tyrosine |
| | kinase (Tyro3), mRNA |
| D37880 | Rat mRNA for Sky, complete cds |

G. Axl: AXL receptor tyrosine kinase
(aliases; ufo oncogene homolog, Ark, Ufo, axl, Tyro7)
Mouse Axl mRNA SEQUENCES (2)

| | |
|---|---|
| X63535 | M. musculus ufo mRNA |
| NM_009465 | Mus musculus AXL receptor tyrosine kinase (Axl), mRNA |

Human Axl mRNA SEQUENCES (7)

| | |
|---|---|
| NM_001699 | Homo sapiens AXL receptor tyrosine kinase (AXL), transcript |
| | Variant 2, mRNA |
| X57019 | H. sapiens mRNA for tyrosine kinase receptor |
| BC032229 | Homo sapiens, clone MGC:34202 IMAGE:5205825, mRNA, |
| | complete cds |
| S65125 | UFO = proto-oncogene [human, NIH3T3 cell, mRNA, 3116 nt] |
| M76125 | Human tyrosine kinase receptor (axl) mRNA, complete cds |
| NM_021913 | Homo sapiens AXL receptor tyrosine kinase (AXL), transcript |
| | Variant 1, mRNA |
| X66029 | H. sapiens mRNA for tyrosine kinase receptor |

H. Mer-c-mer proto-oncogene
a. (Aliases for mouse; c-mer proto-oncogene, Tyro12, Eyk, Nyk)
Mouse c-Mer mRNA SEQUENCES (3)

| | |
|---|---|
| U21301 | Mus musculus c-mer tyrosine kinase receptor mRNA, |
| | complete eds |
| NM_008587 | Mus musculus c-mer proto-oncogene (Mer), mRNA |

b. (Preferred symbol for human: MERTK
(Aliases for human: MER, C-MER, c-mer)
Human MERTK mRNA SEQUENCES (2)

| | |
|---|---|
| NM_006343 | Homo sapiens c-mer proto-oncogene tyrosine kinase |
| | (MERTK), mRNA |
| U08023 | Human cellular proto-oncogene (c-mer) mRNA, complete |
| | cds |

We claim:

1. A method of alleviating a symptom of a disease or disorder of the nervous system comprising administering Reelin, Gas6, Protein S, a molecule that regulates the phosphorylation status of Dab1 or a combination thereof to modulate neural stem cell or neural progenitor cell activity in vivo to a patient suffering from the disease or disorder of the nervous system.

2. The method of claim 1 wherein the neural stem cell or neural progenitor cell activity is proliferation, differentiation, migration or survival.

3. The method of claim 1 wherein the activity regulated by Gas6 is proliferation, differentiation or survival.

4. The method of claim 1 wherein the activity regulated by Reelin is proliferation.

5. The method of claim 1 wherein the activity regulated by Protein S is proliferation, differentiation or survival.

6. The method of claim 1 wherein the Reelin, Gas6, Protein S, a molecule that regulates the phosphorylation status of Dab1 or combination thereof is administered in an amount of 0.001 ng/kg/day to 10 mg/kg/day.

7. The method of claim 6 wherein the Reelin, Gas6, Protein S, a molecule that regulates the phosphorylation status of Dab1 or combination thereof is adminstered preferably in an amount of 0.01 ng/kg/day to 5 mg/kg/day.

8. The method of claim 6 wherein the Reelin, Gas6, Protein S, a molecule that regulates the phosphorylation status of Dab1 or combination thereof is administered more preferably in an amount of 0.1 ng/kg/day to 1 mg/kg/day.

9. The method of claim 6 wherein the Reelin, Gas6, Protein S, a molecule that regulates the phosphorylation status of Dab1 or combination thereof is administered most preferably in an amount of 0.1 ng/kg/day to 1 μg/kg/day.

10. The method of claim 1 wherein the Reelin, Gas6, Protein S, a molecule that regulates the phosphorylation status of Dab1 or combination thereof is administered to achieve a target tissue concentration of 0.01 nM to 30 nM.

11. The method of claim 10 wherein the tissue is selected from the group consisting of the volume adjacent to the lateral wall, hippocampus, alveus, striatum, substantia nigra, retina, nucleus basalis of Meynert, spinal cord and cortex.

12. The method of claim 10 wherein the tissue is any region of tissue that is impaired by stroke injury or ischemic injury.

13. The method of claim 1 wherein the Reelin, Gas6, Protein S, a molecule that regulates the phosphorylation status of Dab1 or a combination thereof is administered by injection.

14. The method of claim 13 wherein the injection is given subcutaneously, intraperitoneally, intramusclularly, intracerebroventricularly, intraparenchymally, intrathecally or intracranially.

15. The method of claim 1 wherein the Reelin, Gas6, Protein S, a molecule that regulates the phosphorylation status of Dab1 or combination thereof is administered orally.

16. The method of claim 1 wherein the Reelin, Gas6, Protein S, a molecule that regulates the phosphorylation status of Dab1 or a combination thereof is administered to the buccal, nasal or rectal mucosa.

17. The method of claim 1 wherein the Reelin, Gas6, Protein S, a molecule that regulates the phosphorylation status of Dab1 or combination thereof is administered via peptide fusion or micelle delivery.

18. The method of claim 1 wherein the disease or disorder of the nervous system is selected from the group consisting of neurodegenerative disorders, neural stem cell disorders, neural progenitor disorders, ischemic disorders, neurological traumas, affective disorders, neuropsychiatric disorders, degenerative diseases of the retina, retinal injury/trauma and learning and memory disorders.

19. A method of modulating the activity of a Reelin receptor, Gas 6 receptor, a Protein S receptor or a combination thereof, on a neural stem cell or neural progenitor cell, the method comprising exposing the cell expressing the receptor to exogenous reagent, antibody, or affibody, wherein the exposure induces or inhibits the neural stem cell or neural progenitor cell to proliferate, differentiate or survive.

20. The method of claim 19 wherein the activity regulated by Gas6 is proliferation, differentiation or survival.

21. The method of claim 19 wherein the activity regulated by Reelin is proliferation.

22. The method of claim 19 wherein the activity regulated by Protein S is proliferation, differentiation or survival.

23. The method of claim 19 wherein the Reelin receptor is VLDLR or ApoER2.

24. The method of claim 19 wherein the Gas6 receptor is Axl, Tyro3 or Mer.

25. The method of claim 19 wherein the Protein S receptor is Tyro3.

26. The method of claim 19 wherein the Reelin, Gas6 or Protein S receptor is a fragment of the full length protein.

27. The method of claim 19 wherein the reagent is Reelin, Gas6, Protein S or a molecule that regulates the phosphorylation status of Dab1.

28. The method of claim 19 wherein the antibody is a monoclonal or a polyclonal antibody.

29. The method of claim 19 wherein the neural stem cell or neural progenitor cell is derived from fetal brain, adult brain, neural cell culture or a neurosphere.

30. The method of claim 19 wherein the neural stem cell or neural progenitor cell is derived from tissue enclosed by dura mater, peripheral nerves or ganglia.

31. The method of claim 19 wherein the neural progenitor cell is derived from stem cells originating from a tissue selected from the group consisting of pancreas, skin, muscle, adult bone marrow, umbilical cord tissue and umbilical cord blood.

32. A method for reducing a symptom of a disease or disorder of the central nervous system in a mammal in need of such treatment comprising administering Reelin, Gas6, Protein S, a molecule that regulates the phosphorylation status of Dab1 or a Reelin, Gas6 or Protein S agonist or antagonist to the mammal.

33. The method of claim 32 wherein the Reelin, Gas6, Protein S, a molecule that regulates the phosphorylation status of Dab1 or a Reelin, Gas6 or Protein S agonist or antagonist is administered in an amount of 0.001 ng/kg/day to 10 mg/kg/day.

34. The method of claim 33 wherein the Reelin, Gas6, Protein S, a molecule that regulates the phosphorylation status of Dab1 or a Reelin, Gas6 or Protein S agonist or antagonist is adminstered preferably in an amount of 0.01 ng/kg/day to 5 mg/kg/day.

35. The method of claim 33 wherein the Reelin, Gas6, Protein S, a molecule that regulates the phosphorylation status of Dab1 or or a Reelin, Gas6 or Protein S agonist or antagonist is administered more preferably in an amount of 0.1 ng/kg/day to 1 mg/kg/day.

36. The method of claim 33 wherein the Reelin, Gas6, Protein S, a molecule that regulates the phosphorylation status of Dab1 or or a Reelin, Gas6 or Protein S agonist or antagonist is administered most preferably in an amount of 0.1 ng/kg/day to 1 μg/kg/day.

37. The method of claim 32 wherein the Reelin, Gas6, Protein S, a molecule that regulates the phosphorylation status of Dab1 or combination thereof is administered to a tissue in a concentration of 0.01 nM to 30 nM.

38. The method of claim 37 wherein the tissue is selected from the group consisting of the volume adjacent to the lateral wall, hippocampus, alveus, striatum, substantia nigra, retina, nucleus basalis of Meynert, spinal cord and cortex.

39. The method of claim 37 wherein the tissue is any region of tissue that is impaired by stroke injury or ischemic injury.

40. The method of claim 32 wherein the Reelin, Gas6 or Protein S agonist or antagonist is selected from the group consisting of an antibody, an affibody, a small molecule and a receptor.

41. The method of claim 40 wherein the receptor is a Reelin receptor, Gas6 receptor, Protein S or combination thereof.

42. The method of claim 41 wherein the Reelin receptor is VLDLR or ApoER2.

43. The method of claim 41 wherein the Gas6 receptor is Axl, Tyro3 or Mer.

44. The method of claim 41 wherein the Protein S receptor is Tyro3.

45. The method of claim 32 wherein the administration is local or systemic.

46. The method of claim 32 further comprising administering a ventricle wall permeability enhancer.

47. The method of claim 46 wherein the ventricle wall permeability enhancer is administered before, during or after administration of Reelin, Gas6, Protein S or a molecule that regulates the phosphorylation status of Dab1 or a Reelin, Gas6 or Protein S agonist or antagonist.

48. The method of claim 46 wherein the ventricle wall permeability enhancer or the Reelin, Gas6, Protein S, molecule that regulates the phosphorylation status of Dab1 or Reelin, Gas6 or Protein S agonist or antagonist are admixed with a pharmaceutically acceptable carrier.

49. The method of claim 46 further comprising administration of one or more agents selected from the group consisting of stem cell mitogens, survival factors, glial-lineage preventing agents, anti-apoptotic agents, anti-stress medications, neuroprotectants, anti-pyrogenics, differentiation factors and a combination thereof.

50. A method for inducing the in situ proliferation, differentiation or survival of a neural stem cell or neural progenitor cell located in the neural tissue of a mammal, the method comprising administering a therapeutically effective amount of Reelin, Gas6, Protein S or a molecule that regulates the phosphorylation status of Dab1 to the neural tissue to modulate the proliferation, differentiation or survival of the cell.

51. The method of claim 50 wherein a reagent is used to modulate the proliferation, differentiation or survival of the cell.

52. The method of claim 50 wherein the reagent is selected from the group consisting of an antibody, an affibody, a small molecule and a receptor.

53. The method of claim 50 wherein the administration of the Reelin, Gas6, Protein S or molecule that regulates the phosphorylation status of Dab1 is systemic or local.

54. The method of claim 50 wherein the administration of the Reelin, Gas6, Protein S or a molecule that regulates the phosphorylation status of Dab1 alleviates a symptom of a diseases or disorders of the nervous system.

55. The method of claim 54 wherein the disease or disorder of the nervous system is selected from the group consisting of neurodegenerative disorders, neural stem cell disorders, neural progenitor disorders, ischemic disorders, neurological traumas, affective disorders, neuropsychiatric disorders, degenerative diseases of the retina, retinal injury/trauma and learning and memory disorders.

56. The method of claim 50, further comprising administering a ventricle wall permeability enhancer.

57. The method of claim 56 wherein the ventricle wall permeability enhancer is administered before, during, or after administration of the reagent.

58. The method of claim 56 wherein the ventricle wall permeability enhancer and the Reelin, Gas6, Protein S or a molecule that regulates the phosphorylation status of Dab1 are admixed with a pharmaceutically acceptable carrier.

59. The method of claim 50 further comprising administration of one or more agents selected from the group consisting of stem cell mitogens, survival factors, glial-lineage preventing agents, anti-apoptotic agents, anti-stress medications, neuroprotectants, anti-pyrogenics, differentiation factors and a combination thereof.

60. A method for accelerating the growth of neural stem cells or neural progenitor cells in a desired target tissue in a subject, comprising administering to the subject an expression vector containing a Reelin, Gas6 or Protein S gene in a therapeutically effective amount.

61. The method of claim 60 wherein the expression vector is administered by injection.

62. The method of claim 61 wherein the injection is given subcutaneously, intraperitoneally, intramuscluarly, intracerebroventricularly, intraparenchymally, intrathecally or intracranially.

63. The method of claim 60 wherein the expression vector is administered orally.

64. The method of claim 60 wherein the expression vector is administered via peptide fusion or micelle delivery.

65. The method of claim 60 wherein the expression vector is administered to the buccal, nasal or rectal mucosa.

66. The method of claim 60 wherein the expression vector is a non-viral expression vector encapsulated in a liposome.

67. A method of enhancing neurogenesis in a patient suffering from a disease or disorder of the central nervous system, by infusion of Reelin, Gas6, Protein S, a molecule that causes the regulates the phosphorylation status of Dab1 or a Reelin, Gas6 or Protein S receptor agonist or antagonist.

68. The method of claim 67 wherein the infusion is selected from the group consisting of intraventricular, intravenous, sublingual, subcutaneous and intraarterial infusion.

69. The method of claim 67 wherein the disease or disorder of the central nervous system is selected from the group consisting of neurodegenerative disorders, neural stem cell disorders, neural progenitor disorders, ischemic disorders, neurological traumas, affective disorders, neuropsychiatric disorders, degenerative diseases of the retina, retinal injury/trauma and learning and memory disorders.

70. A method of alleviating a symptom in a patient suffering from a disease or disorder of the central nervous system by enhancing neurogenesis through infusion of Reelin, Gas6, Protein S, a molecule that regulates the phosphorylation status of Dab1 or a Reelin, Gas6 or Protein S receptor agonist or antagonist.

71. The method of claim 70 wherein the infusion is selected from the group consisting of intraventricular, intravenous, sublingual, subcutaneous and intraarterial infusion.

72. The method of claim 70 wherein the disease or disorder of the central nervous system is selected from the group consisting of neurodegenerative disorders, neural stem cell disorders, neural progenitor disorders, ischemic disorders, neurological traumas, affective disorders, neuropsychiatric disorders, degenerative diseases of the retina, retinal injury/trauma and learning and memory disorders.

73. A method for producing a population of cells enriched for human neural stem cells or human neural progenitor cells, comprising:
a. contacting a population containing neural stem cells or neural progenitor cells with a reagent that recognizes a determinant on a Reelin receptor, Gas6 receptor or Protein S receptor; and
b. selecting for cells in which there is contact between the reagent and the determinant on the surface of the cells of step (a), to produce a population highly enriched for central nervous system stem cells.

74. The method of claim 73 wherein the reagent is a reagent selected from the group consisting of a soluble receptor, a small molecule, a peptide, an antibody and an affibody.

75. The method of claim 74 wherein the soluble receptor is a Reelin, Gas6 or Protein S receptor.

76. The method of claim 75 wherein the Reelin receptor is VLDLR or ApoER2.

77. The method of claim 75 wherein the Gas6 receptor is a Axl, Tyro3, Mer or a combination thereof.

78. The method of claim 75 wherein the Protein S receptor is Tyro3.

79. The method of claim 74 wherein the antibody is a monoclonal or a polyclonal antibody.

80. The method of claim 73 wherein the population containing neural stem cells or neural progenitor cells are obtained from any population of cells which gives rise to neural tissue.

81. The method of claim 80 wherein the population of cells is fetal brain or adult brain.

82. The method of claim 73 wherein the human neural progenitor cells are derived from stem cells originating from a tissue selected from the group selected from pancreas, skin, muscle, adult bone marrow, umbilical cord tissue and umbilical cord blood.

83. An in vitro cell culture comprising a cell population generated by the method of claim 73 wherein the cell population is enriched in receptor expressing cells wherein the receptors are selected from the group consisting of VLDLR, ApoER2, Axl, Tyro3 and Mer receptor.

84. A method for alleviating a symptom of a disease or disorder of the central nervous system comprising administering the population of claim 83 to a mammal in need thereof.

85. A non-human mammal engrafted with the human neural stem cells or neural progenitor cells of claim 83.

86. The nonhuman mammal of claim 85 wherein the non-human mammal is selected from the group including rat, mouse, rabbit, horse, sheep, pig and guinea pig.

87. A method of reducing a symptom of a disease or disorder of the central nervous system in a subject comprising the steps of administering into the spinal cord of the subject a composition comprising a population of isolated neural stem cells or neural progenitor cells obtained from fetal or adult tissue; and Reelin, Gas6, Protein S, a molecule that regulates the phosphorylation status of Dab1 or a Reelin, Gas6 or Protein S agonist or antagonist or a combination thereof such that the symptom is reduced.

88. The method of claim 87 wherein the disease or disorder of the central nervous system is selected from the group consisting of neurodegenerative disorders, neural stem cell disorders, neural progenitor cell disorders, ischemic disorders, neurological traumas.

89. A method of gene delivery and expression in a target cell of a mammal, comprising the step of introducing a viral vector into the target cell, wherein the viral vector has at least one insertion site containing a nucleic acid which encodes Reelin, Gas6, Protein S, a Reelin receptor, a Gas6 receptor or a Protein S receptor, the nucleic acid gene operably linked to a promoter capable of expression in the host.

90. The method of claim 89 wherein the Reelin receptor is VLDLR or ApoER2.

91. The method of claim 89 wherein the Gas6 receptor is Axl, Tyro3, Mer or a combination thereof.

92. The method of claim 89 wherein the Protein S receptor is Tyro3.

93. The method of claim 89 wherein the viral vector is a non-lytic viral vector.

94. A method of gene delivery and expression in a target cell of a mammal comprising the steps of:
(a) providing an isolated nucleic acid fragment of a nucleic acid sequence which encodes for Reelin, Gas6, Protein S, Dab1, a molecule that regulates the phosphorylation status of Dab1, a Reelin receptor, a Gas6 receptor or a Protein S receptor;
(b) selecting a viral vector with at least one insertion site for insertion of the isolated nucleic acid fragment operably linked to a promoter capable of expression in the target cells;
(c) inserting the isolated nucleic acid fragment into the insertion site, and
(d) introducing the vector into the target cell wherein the gene is expressed at detectable levels.

95. The method of claim 94 wherein the Reelin receptor is VLDLR or ApoER2.

96. The method of claim 94 wherein the Gas6 receptor is Axl, Tyro3, Mer or a combination thereof.

97. The method of claim 94 wherein the Protein S receptor is Tyro3.

98. The method of claim 94 wherein the virus is selected from the group consisting of retrovirus, adenovirus, pox virus, iridoviruses, coronaviruses, togaviruses, caliciviruses, lentiviruses, adeno-associated viruses and picornaviruses.

99. The method of claim 98 wherein the pox virus is vaccinia.

100. The method of claim 94 wherein the virus is a strain that has been genetically modified or selected to be non-virulent in a host.

101. A method for alleviating a symptom of a disease or disorder of the central nervous system in a patient comprising the steps of:
(a) providing a population of neural stem cells or neural progenitor cells;
(b) suspending the neural stem cells or neural progentor cells in a solution comprising Reelin, Gas6, Protein S or a molecule that regulates the phosphorylation status of Dab1 or a combinatin thereof to generate a cell suspension;
(c) delivering the cell suspension to an injection site in the central nervous system of the patient to alleviate the symptom.

102. The method of claim 101 further comprising the step of injecting the injection site with a growth factor for a period of time before the step of delivering the cell suspension.

103. The method of claim 102 further comprising the step of injecting the injection site with the growth factor after said delivering step.

104. A method for transplanting a population of cells enriched for human neural stem cells or human neural progenitor cells, comprising:
(a) contacting a population containing neural stem cells or neural progenitor cells with a reagent that recognizes a determinant on a Reelin receptor, Gas6 receptor or Protein S receptor;
(b) selecting for cells in which there is contacted between the reagent and the determinant on the surface of the cells of step (a), to produce a population highly enriched for central nervous system stem cells; and
(c) implanting the selected cells of step (b) into a non-human mammal.

105. A method of modulating a Reelin, Gas6 or Protein S receptor or a Reelin, Gas6 or Protein S ligand on the surface of a neural stem cell or neural progenitor cell comprising the step of contacting the cell expressing the receptor, or ligand to exogenous reagent, antibody, or affibody, wherein the exposure induces or inhibits the neural stem cell or neural progenitor cell to proliferation, differentiation or survival.

106. The method of claim 105 wherein the antibody is a monoclonal or a polyclonal antibody.

107. The method of claim 105 wherein the neural stem cell or neural progenitor cell is derived from fetal brain, adult brain, neural cell culture or a neurosphere.

108. A method of determining an isolated candidate Reelin, Gas6 or Protein S receptor modulator compound for its ability to modulate neural stem cell or neural progenitor cell activity comprising the steps of:
(a) administering the isolated candidate compound to a non-human mammal; and
(b) determining if the candidate compound has an effect on modulating the neural stem cell or neural progenitor cell activity in the non-human mammal.

109. The method of claim 108 wherein said determining step comprises comparing the neurological effects of said non-human mammal with a referenced non-human mammal not administered the candidate compound.

110. The method of claim 108 wherein the compound is selected from the group consisting of a peptide, a small molecule, a soluble receptor a receptor agonist and a receptor antagonist.

111. The method of claim 108 wherein the compound is selected from the group consisting of VLDLR, ApoER2, Axl, Mer, Tyro3, a soluble fragment thereof and an extracellular fragment thereof.

112. The method of claim 108 wherein the neural stem cell or neural progenitor cell activity is proliferation, differentiation, migration or survival.

113. The method of claim 108 wherein the Reelin, Gas6 or Protein S receptor modulator is administered by injection.

114. The method of claim 113 wherein the injection is given subcutaneously, intraperitoneally, intramuscluarly, intracerebroventricularly, intraparenchymally, intrathecally or intracranially.

115. The method of claim 108 wherein the Reelin, Gas6 or Protein S receptor modulator is administered via peptide fusion or micelle delivery.
